SEQUENCE LISTING (24 sequences)

SEQ ID NO: 1 - DNA, 201 bp, Oryza sativa
  rice Oryza sativa ssp. japonica strain Zhonghua 11 PCR product fragment 1 for RNA interference vector
ggctgcacggatagagttccagaagaataggttcagaacaagacacacctccgatgttttggctcgcatgcagttagctaaggcgaacaacttcagtattgatctacagaaagaaaaagatggaaaccccataaatattgacacagctacagtatcagatatactgaagaaggcactcagttatttctcagccatacaggc

SEQ ID NO: 2 - protein, 785 aa, Oryza sativa
  rice Oryza sativa strain OsOSC8 triterpene synthase
MWKLKIAEGGPWLKSGNSHVGRETWEFDPNFGTSEEREAVEAARIEFQKNRFRTRHTSDVLARMQLAKANNFSIDLQKEKDGNPINIDTATVSDILKKALSYFSAIQAYDGHWPGDFPGPLFTTATMIIVLYVTESLTITLSSEHHKEICRYLYNRQNIDGGWGLHAEGESSMLSTALNYTALRLLGENVDDGPDISMHKARKWIHDHGGATMIPILGKVWLSVLGVFDWSGVNPIPPELFLLPSFVPIQPGRLWSHFRMAFIPMSYLYGKKFVGPITRLVISLREELHIHPYKKIDWKEARKLCAKEDAYNPHMWLQECLSDCLYSFGEPFLTRWPISYMRKRALYQIAEFLKYEDENSQYICIGAAQKALSMLCCWIENPNSDAFKRHLARVADFLWVGEDGMKVRVCAGQLWDVAFAVQAILACSIAEEFGSTLKKAHGFIKTSQIMDNPSGDFSRKYRHISKGGWAFQVADQGWQVSDCTAEALKALLLLSKCLSDGADYQMETYCYFDAVNVLLSLQNPNGGYGAWELARTYPWMEIFNMTEIYADIIVEHQYVECTSSVIQALALFREKYPGHRKDEIDQCIRKATEFIEKLQNDDGSWFGSWGICFTYGTWFAIEGLSAVGQCYDDSTCIRKACKFLLSKQLTNGGWGESHLSSRTKAYTNLDGEKSHIVNTAWAMLALMKAGQVERDPAPLHKAARLIMSMQLSDGDFPQEEMIGSFLKNGPLCYMAYRNIFPIWALGEYQKLVFQNYQTSSIKQTNIAPSAGNAALKNSASTTAPT

SEQ ID NO: 3 - DNA, 30 bp, Artificial Sequence
  synthetic PCR amplification Primer pair 1 sense primer
aaaaagcaggctggctgcacggatagagtt

SEQ ID NO: 4 - DNA, 30 bp, Artificial Sequence
  synthetic PCR amplification Primer pair 1 antisense primer
agaaagctgggtgcctgtatggctgagaaa

SEQ ID NO: 5 - DNA, 2358 bp, Oryza sativa
  rice Oryza sativa strain OsOSC8 triterpene synthase
atgtggaagctcaagattgccgagggaggaccatggctaaagagtggaaacagtcatgttggaagagaaacatgggaatttgacccaaattttggaacaagtgaagagcgggaggcggttgaggctgcacggatagagttccagaagaataggttcagaacaagacacacctccgatgttttggctcgcatgcagttagctaaggcgaacaacttcagtattgatctacagaaagaaaaagatggaaaccccataaatattgacacagctacagtatcagatatactgaagaaggcactcagttatttctcagccatacaggcatatgatgggcactggccaggggatttcccaggcccactgtttaccactgcaaccatgatcatagttctatatgtcacagagtcattaactattacactgtcatcggaacatcacaaggagatctgtcgatatctgtacaatcgtcagaacatcgatggaggatggggactacatgcagaaggtgaaagttccatgcttagcacagctctcaattatactgctctgagactacttggggagaatgttgatgatggaccagatatatccatgcataaagcaagaaaatggatacatgaccatggaggtgcaacaatgataccgatcttgggaaaagtgtggctctcggtgcttggagtttttgactggtcaggtgtaaatcctattcccccagaattatttcttcttccatctttcgttcctatccaaccaggacgattatggagtcacttccgaatggcttttattcccatgtcttatttgtatggaaaaaagtttgtcggtcctataacaagattggttatatcattaagggaagagctacatattcatccctataaaaagattgactggaaggaggcacgcaaattatgtgcaaaggaagatgcctataatccacatatgtggctacaagagtgcctatctgactgcctttatagttttggtgagccgtttcttacacgttggccaatttcctacatgagaaaaagagctctataccaaattgctgagttcttgaagtatgaagatgaaaattctcagtatatctgcattggtgctgcacagaaggcattatccatgttgtgctgttggattgaaaatcccaattcagatgcattcaagcgtcatttggctagagtagctgattttctatgggttggcgaagatggcatgaaagttcgggtctgtgcgggtcaattatgggatgttgctttcgcagtccaagcgatattagcgtgtagcatcgcagaagaatttggaagtacccttaagaaagcacatggtttcataaaaacttcccagattatggacaatccttctggtgacttcagcagaaagtaccgtcacatatctaaaggaggatgggccttccaggttgcagatcaggggtggcaggtttcagattgcacagcagaagctcttaaggctttgctactgctgtcaaagtgtttgtcagatggtgcagattatcaaatggaaacctactgctactttgacgcggtgaatgtattactctcgttacagaacccaaatggtgggtatggagcatgggagctagctcgcacatatccatggatggagattttcaacatgacagagatatatgcagacatcattgtggagcatcagtacgtagagtgtacctcatcagtcatccaagcattggcattgttccgggagaagtaccctgggcatcggaaagatgaaatagatcaatgcatcaggaaagcaacagaattcatcgagaagttacagaatgatgatggatcatggtttggatcatggggtatttgcttcacgtatggaacatggtttgcaatagagggtctgtcagccgtggggcagtgttatgatgacagcacctgcattcggaaagcctgtaagtttctgttatcaaagcaactaacaaatggtggatggggggagagtcatctttcatccagaaccaaggcatacacaaacctagatggggagaaatcacatatagtcaatactgcatgggcaatgttggcactcatgaaagctggacaggtagagagagaccccgctcctctgcacaaagcagcaagacttatcatgagcatgcagctcagtgatggtgactttccacaagaggaaatgatcggaagtttcttgaaaaatggccccttgtgttatatggcttatcgcaatatattcccaatatgggctcttggagagtatcagaaattagtatttcagaactatcagacctctagcatcaagcaaacaaatatagctccatctgctggcaatgctgctctgaagaattcagcaagtacaacagcgccaacttga

SEQ ID NO: 6 - DNA, 25 bp, Artificial Sequence
  synthetic PCR amplification Primer pair 2 sense primer OsOSC8T1F
gaggtcaagtcgtcttctgcaatta

SEQ ID NO: 7 - DNA, 22 bp, Artificial Sequence
  synthetic PCR amplification Primer pair 2 antisense primer OsOSC8T1R
atttgtctgcgctctgcacatg

SEQ ID NO: 8 - DNA, 25 bp, Artificial Sequence
  synthetic PCR amplification Primer pair 3 sense primer OsOSC8T13F
gcttaaaggtaaatttcaggcttcc

SEQ ID NO: 9 - DNA, 25 bp, Artificial Sequence
  synthetic PCR amplification Primer pair 3 antisense primer OsOSC8T13R
cgatcagaatcaattaaacccagac

SEQ ID NO: 10 - DNA, 25 bp, Artificial Sequence
  synthetic PCR amplification Primer pair 4 sense primer OsOSC8T17F
tcatccttagattaattagccgaca

SEQ ID NO: 11 - DNA, 25 bp, Artificial Sequence
  synthetic PCR amplification Primer pair 4 antisense primer OsOSC8T17R
cataaggatctcataaaatcgacca

SEQ ID NO: 12 - DNA, 20 bp, Artificial Sequence
  synthetic RT-PCR amplification 5′ primer for TaOSC1
atgtggaagctcaagatcgc

SEQ ID NO: 13 - DNA, 22 bp, Artificial Sequence
  synthetic RT-PCR amplification 3′ primer for TaOSC1
ttagccagagcaaagtactaat

SEQ ID NO: 14 - DNA, 2280 bp, Triticum aestivum
  Chinese spring wheat Triticum aestivum triterpene synthase TaOSC1
atgtggaagctcaagatcgccgagggcggcccgtggctcacgagcggcaacaaccacttcggaagagaaacatgggagtttgaccgaaatgacgctggatcaagcgaagagcgggatgcggtcgacgctgcacgggctgaattccagaagaacaggttcaggacaaggcacagctccgatgttttggcccgcatgcagttagtaaaggggaataacttcagccttgaccaacaacagaaaccaaaaggtgatgaaactagtgtcgacatcaacatagctacggtgtcggagacactgaaaagggcactcagatacttctcagccatacaagcgcacgatgggcactggccaggagattttccgggccctctctttaccacagcaaccatgatcgtagttttgtatgtcacggagtcgttaggtactacgctatcgtcagaacaccgcaaggagatctgtcgctatttgtacaaccgacagaatatggatggaggatggggactacatgcggaaggcgagagctccatgctcagctcagctctcaactacgctgctctaagactacttggtgagggtgctgatgatggaccagatatgtccatgccaaaagctaggaaatggatacatgaccatggtggtgcaacaatgataccaatattgggaaaagtgtggctttcggtacttggggtttctgaatggtcaggtgtaaaccctatgcccccagaattgttccttctgccatccttcgttcctatccaaccaggacggttgtggagtcacttcagaatggctttcatccccatgtcctatttatatggcaagaagtttgttggcccaataacaaaactggttttatcattaagggaagagctgcatattcacccctacaaaaagattaactggaagcaagcgcgcaaattatgcgcaaaggaagatgcgtatcatccacatacctggctccaagaatgcttgtccgattgcctttacagtttcggtgaaccttttctggcatgttggccagtttcccacatgagacgaaaagctctacgacaaattgccgatttcctgaaatacgaagatgatatttcacggtatatctgcattggcgctgcgcaaaaggcattatccatgttatgctgttggtctgagaatcccagttcagatgcattcaagcgccacttggctagagtttctgatttcctatggctcggtgaagatggcatgaaagtgcgggtatgtgcaggtcagtcatgggatgttgcttttgctgtacaagcaatattagcgtgtgatgttgcacaggaatttggaactactctgaagaaagcacaccatttcataaaagcatcccagattgtcagcaatcctaccggtgacttcagcagaaagtaccgtcacatctctaaaggaggatgggccttccaggttgcagaccagggctggcaggtttcagactgcacagcagaagctctcaaggctctgttactgctctcaaagtttccgtcagagatcgtgggtgatcagatggaaacatgccgcttccatgatgcagtgaacatattattatctttacagaatcctaatggtggctatggaacttgggagctagctcgtacatatccatggatggagaatttaaacatgacagagatatatgcagacatcatggtggagcatcagtacgtcgagtgtacctcgtcggtcatccaagcattggccctgtttcggcaaaaataccccgggcatcgggaagatgaagtagaacaatgcatcaggagagcgacagaattcatcgagaagttacagaatgaggacggttcatggttcggatcatggggtatttgcttcacatacggcacgtggtttgctatagagggcctatcggcagttggacagtgttacaataatagcacctacatccggaagggttgccagtttctattatcaaagcagctaaggaatggtggatggggtgagagtcatctttcatccacaaccaaggcatacacgaacctagacggggagaaatcacatgtagtcaacaccgcatgggcaatgttggcactaatgaaagctggacaggccgaacgagatccgtctcctttgcacgaagctgcaagacttatcatgagcatgcaacttggcaatggtgacttcccacaggaggaaatgattggaagtttcttgaaaaatggtcccttgtgttatatggcttatcgcaacatattccccatatgggcccttggagagtatcatagattagtactttgctctggctaa

SEQ ID NO: 15 - protein, 759 aa, Triticum aestivum
  Chinese spring wheat Triticum aestivum triterpene synthase TaOSC1
MWKLKIAEGGPWLTSGNNHFGRETWEFDRNDAGSSEERDAVDAARAEFQKNRFRTRHSSDVLARMQLVKGNNFSLDQQQKPKGDETSVDINIATVSETLKRALRYFSAIQAHDGHWPGDFPGPLFTTATMIVVLYVTESLGTTLSSEHRKEICRYLYNRQNMDGGWGLHAEGESSMLSSALNYAALRLLGEGADDGPDMSMPKARKWIHDHGGATMIPILGKVWLSVLGVSEWSGVNPMPPELFLLPSFVPIQPGRLWSHFRMAFIPMSYLYGKKFVGPITKLVLSLREELHIHPYKKINWKQARKLCAKEDAYHPHTWLQECLSDCLYSFGEPFLACWPVSHMRRKALRQIADFLKYEDDISRYICIGAAQKALSMLCCWSENPSSDAFKRHLARVSDFLWLGEDGMKVRVCAGQSWDVAFAVQAILACDVAQEFGTTLKKAHHFIKASQIVSNPTGDFSRKYRHISKGGWAFQVADQGWQVSDCTAEALKALLLLSKFPSEIVGDQMETCRFHDAVNILLSLQNPNGGYGTWELARTYPWMENLNMTEIYADIMVEHQYVECTSSVIQALALFRQKYPGHREDEVEQCIRRATEFIEKLQNEDGSWFGSWGICFTYGTWFAIEGLSAVGQCYNNSTYIRKGCQFLLSKQLRNGGWGESHLSSTTKAYTNLDGEKSHVVNTAWAMLALMKAGQAERDPSPLHEAARLIMSMQLGNGDFPQEEMIGSFLKNGPLCYMAYRNIFPIWALGEYHRLVLCSG

SEQ ID NO: 16 - DNA, 2280 bp, Hordeum vulgare
  barley Hordeum vulgare strain Varda triterpene synthase HvOSC1
atgtggaaactcaagatcgctgagggtggcccgtggctcacgagtggcaacaatcatgccggaagagaaacatgggagtttgaccaaaacgacgccggatcaagcgaagaccgggatgcggtcgatgctgcacggtctgaattccagaagaacaggtttaggacaaggcacagctctgatgttttggctcgcctgcagctagcaaaggagaataacttcagccttgaccaaaaacagaaaccaaaagatgatgaaactagtgtcgttatcaatgtagctacggtgtcggagacactggaaagggcactcggatacttctcggccatacaagcgcatgatgggcactggccaggagattttccggggccactctttaccacagcaaccatgatcatagttttgtatgtcacggagtcgttaggtagtacgctatcatcagaacatcgcaaggagatctgtcgctatttgtacaaccgtcagaatatagatgggggatggggactacacgcggaaggcgaaagctccatgctcagctcagctctcaactacactgctctaagactgcttggcgagggtgttgatgatggaccagacatgtccatgccgaaagcaaggaaatggatacatgaccatggcggtgcaacgatggtaccaatcttgggaaaagtgtggctctcggtgcttggagttttcgaatggtcaggtgtaaaccctattcccccagaattgttccttctgccatcctttgttcctattcaaccaggaagattgtggagccacttcagaatggctttcattcccatgtcctatttgtatggcaagaaatttgttggcccaataaccaaattagttttgtcattaagagaagagctgcatattcatccctacaaaaagattaactggaggcaagcacgcaaattatgtgcaaaggaagacgcctatcacccacacacctggcttcaagaatgcttgtctgactgcctttacagttttggcgaaccttttctggcacgttggccggtttcctacatgagaagaagagctctacgacaaattgccgagttcctgaaatacgaagatgacaactcacggtatatctgcatcggcgccgcgcaaaaggcactatccatgttatgctgttggtctgagaatcccaattcagatgcattcaagaaccacttggctagagttgctgatttcctgtggctcggcgaagatgggatgaaagttcgggtgtgtgcaggtcaatcatgggatgtcgctttcgccgtgcaagcgatactagcatgtagtgttgcagaggaatttggaagtactctcaagaaagcacatcatttcataaaagcgtcacagattttggacaatccttctggtgatttcggcagaaggtaccgtcacatttctaaaggaggatgggccttccaggttgcagatcagggttggcaggtttcagattgcacagcagaagctcttaaggctctgttactgctctcaaagtttccgtcagatatcgttggcgatcagatggaaacatgccgctaccatgatgcggtgaatgtattactatctttacagaatcctaatggtggctatgggacttgggagctagctcgtacgtatccatggatggagaatttaaacatgacagagatatatgcagacatcatggtggagcatcagtacgtcgagtgcacctcgtcggccatccaggcattggccctgtttcggcaaaaataccccgggcatcgggaagatgaaatagaacagtgcatcaggagagcgacagagttcatcgagaagttacagaatgaggacggttcatggtttggatcatggggtatttgcttcacatatggcacatggttcgctatagagggcctgtcagcagttggacagtgttacgataacagcacctacattcggaaggcttgccagtttctattatcgaagcagctaacgaatggtggatggggtgagagtcatctttcatccacaaccaaggcatacacgaacctagaaggggagaaatcgcatgtagtcaacacggcgtgggcaatgttggcactaatgaaagctggacaggccgaaagagatccatctcctttgcacgaagcagcaagacttatcatgagcatgcagcttggcaatggtgacttcccacaggaggaaatgattggaagtttcttgaaaaatggccccttgtgttatatggcttatcgcaatatattccccatatgggcccttggagagtatcataaattagtactttactctgactaa

SEQ ID NO: 17 - protein, 759 aa, Hordeum vulgare; identical to SEQ ID NO: 15
  barley Hordeum vulgare strain Varda triterpene synthase HvOSC1

SEQ ID NO: 18 - protein, 735 aa, Sorghum bicolor
  Sorghum bicolor triterpene synthase SbOSC1
MWRLKVARGGPWLRSTNGFIGRAVWEFDPDLGTPEERAEVDRVRREFSDRRFHRRESADLLMRMQCAKQKRYQRDLPCLKLEEDENVTEEIVVSSLRRALDQFSSLQASDGHWPGDFSGIMFIMPGLIFALYVTGSMNVVISPEHRREICRYIYNHQNEDGGWGLHIEGHSTMLSSALNYVALRLLGECPNGGDGAMEKGRNWILDHGGAIFMAAWGKFWLSVLGVYDWSGNNPVPPELWLLPHYLPFHPGRVVCYCRMVYMPMSYIYGRRFVGPITPLVLELRKELHTWLQECLSDCLYSFGEPFLTRWPISYMRKKALKQVAEFLKYEDENSQYICIGAAQKALSMLCCWIEKSNSDAFKRHLARVADFLWIGEDGMKVRVCAGQLWDVAFAVQAILACNIAEEYRSTLKRAHDFIKASQIMDNPSGDFSRKYRHISGGWGFQVADQGWQVSDCTAEALKVLLMLSKFSSDIGSDQMETCRLYNAVNVLLSLQNPNGGYGTWELARTYPWMEIFDMTEIYADIMVAHQHVKCTSSVMQALALFKEKYPWHRKDEIDQCIRGATEFIEKLQNDDGSWFGSWGICFTYGTWFAIEGLSAVGQSYGNSTCIRKACKFLLTKQLNNGGWGESYLSSRTKAYTNLDRQKSHIVNTAWAMLALMKAGQVERDPTPLHKAARLIMSMQLGNGDFPQEEMIGSFLKNGPLCYMAYRNIFPIWALGEYQKLVLQCDLQRPTF

SEQ ID NO: 19 - DNA, 2211 bp, Sorghum bicolor
  Sorghum bicolor triterpene synthase SbOSC1
atgtggaggctgaaggtggcgcggggcgggccatggctgcggtcgaccaacggcttcattggacgggcagtgtgggagttcgacccggaccttggcacgccggaggagcgcgccgaggtggacagggtccgccgggagttctccgaccgccgcttccacaggagggagtccgccgacctcctcatgcgcatgcagtgcgcaaagcagaagagatatcaacgtgatctgccgtgcctcaaacttgaggaggatgagaatgtcactgaagaaattgtagtgagctccttgaggcgggctctggatcagttctcttcgctgcaagcaagtgatggtcactggcctggtgatttcagtgggatcatgttcatcatgcctggtttgatatttgccttgtatgtcactggatcaatgaatgttgtcatatcaccggaacatcggcgtgagatatgtcgctatatttacaatcatcagaatgaagatggagggtggggattgcacatcgagggccacagcaccatgctcagctcagccttgaactatgttgctttgagattgcttggggagtgcccaaatggcggagatggagccatggagaaaggccgaaactggatactggatcatggaggagccatatttatggcagcatggggaaagttttggctctcagtactcggagtatatgattggtccggaaataacccggtaccaccagaattgtggctactaccacattacctgccgtttcacccaggtcgagtggtttgctattgccgaatggtgtatatgcccatgtcttacatttatggaaggaggttcgttggccccataacaccactagtactggaattaagaaaagagctacatacatggctacaggagtgtctatctgactgcctttacagttttggtgaaccttttctcacacgttggccaatttcctacatgagaaagaaagccctaaaacaagtggctgaattcttgaaatacgaagatgagaattctcaatacatctgcattggtgccgcacagaaggcattgtccatgttgtgctgttggatcgaaaaatccaactcagatgcattcaagcgccatttggctagagttgctgatttcctatggattggtgaagatggcatgaaagtgcgggtctgtgctggtcaactatgggatgttgcttttgcagtccaagcaatattagcatgtaatattgcagaagaatatagaagcaccctcaagagagcgcatgatttcataaaagcctcccagatcatggacaatccttctggtgacttcagcagaaagtaccgtcacatatctnaaggagggtggggcttccaggttgcagatcaggggtggcaggtttcagattgcacagcagaagctcttaaggttttgttaatgctgtccaaattttcatcagacattggtagtgatcagatggaaacatgccgcttatataatgcagtgaatgtattattatccttacagaacccaaacggtggctatggaacttgggagctagctcgcacatatccatggatggagatttttgacatgacagagatatatgcagatatcatggtggcgcatcagcacgtcaagtgtacctcatcagtcatgcaagcattagcattgttcaaggagaagtacccttggcatcgaaaagatgaaatagatcaatgcattaggggagctactgaattcattgagaagttacagaatgatgatggctcatggtttggatcttggggtatttgtttcacatatggcacatggtttgctattgagggtctatcagctgttggacagagttatggtaacagcacttgtatccggaaggcctgtaagtttctcttaacaaagcagctaaacaatggtggatggggtgagagttatctttcatcaagaaccaaggcatacacaaatctggataggcagaagtcacacatagtgaacactgcatgggcaatgttggcgctcatgaaagctggacaggtggaaagagatcctactccactgcacaaagctgcaagacttatcatgagtatgcagctcggcaatggtgacttcccacaggaggaaatgattggaagtttcttgaaaaatggtcccttgtgctacatggcttatcgtaacatatttcctatatgggcgcttggagagtatcagaaattagtcctccagtgtgacctccaacggccaaccttctag

SEQ ID NO: 20 - protein, 774 aa, Zea mays
  maize Zea mays triterpene synthase ZmOSC1
MWRLKIGEGGPWLKSGNGHIGRETWEFDEDFGSEADREAVDSAREEFSKNRLRMRHSSDLLARMQIAKENGFSLDLHKTRDDDGRSPLLVATTGSSTVSETLRKALDYFAAIQAHDGHWPGDFPGPLFTTATMITVLYVTGSLDSTLSSEHRREICRYLHNRQNADGGWGLHAEGESSMLSTALNYTALRLLGEGGDGGGGPGMSSSSMLIRARKWIRDRGGATMIPILGKVWLSVLGVFEWSGVNPIPPELFLFPSWVPIQPGRLWSHFRMAFIPMSYLYGKKFVGPITRLVVSLREELHVHPYEKIDWKAARNSCAKEDVYSPHTWLQECLSHCLYSFGEPFLTRWPISYMRKKALQQVAEFLKYEDENSQYICIGAAQKALSMLCCWIEKSNSDAFKRHLARVADFLWIGEDGMKVRVCAGQLWDVAFAVQAILACNIAEEYRGTLKKAHDFIKASQIMDNPSGDFSRKYRHISKGGWGFQVADQGWQVSDCTAEALKVLLMLSRFSSDIGSDQMETCRLYDAVNVLLSLQNPNGGYGTWELARTYPWIEIFNMTEIYADIMVEHQYVECTSSVMQALVLFREKDPWHRKDEIDQCIRGATEFIEKLQNDDGSWFGSWGICFTYGTWFAIEGLSAVGQSYGNSTCIQKACKFLLAKQLKNGGWGESHLSSTTKAYTNLDKEKSHIVNTAWAMLALMKAGQAERDPTPLHKAARLIMSMQLSNGDFPQEEMIGSFLKNGPLCYMAYRNIFPIWALGEYQKLVLQCDLQWPTF

SEQ ID NO: 21 - DNA, 2325 bp, Zea mays
  maize Zea mays triterpene synthase ZmOSC1
atgtggcggctgaagatcggcgagggcgggccgtggctcaagagcggcaacgggcacattggaagggagacgtgggagttcgacgaggatttcggatcggaagcagacagggaggccgtcgactccgcgcgggaggagttcagcaagaaccggctccggatgcgacacagctccgatctcttggcccgcatgcagatagcgaaggagaacggtttcagcctcgacctgcacaagacgagagacgacgacgggcggtctcctctgctggtggcgacgacaggctccagcacggtgtcggagacgctgcgaaaggcgctggactacttcgcggcgatacaggcgcacgacgggcactggccaggagacttcccagggccgctgttcaccacagcaaccatgatcacggttctgtacgtgacggggtcgttggacagcacgctgtcgtcggagcaccgcagggagatctgccgctacctgcacaaccgccagaacgcggacggggggtgggggctgcacgcggaaggcgaaagctccatgctcagcacagctctcaactacaccgctctcaggctgctcggcgagggcggagacggcggcggcgggcccggcatgtcgtcgtcgtccatgcttattagagcgaggaagtggatacgtgatcgtggtggcgccaccatgatacccatccttgggaaagtgtggctctcggtgcttggagttttcgaatggtcaggcgtaaaccccatcccaccagaactgtttcttttcccatcctgggttcctattcaaccaggacggctgtggagccacttcaggatggcgttcattcccatgtcctatttgtacggcaagaaatttgtcgggcccatcaccagattggtcgtgtcgctacgggaagagctgcatgtccacccctacgaaaagattgactggaaggcagctcgtaattcatgtgccaaggaagatgtgtatagtccacatacgtggctgcaggagtgtctatctcactgcctttacagcttcggcgaaccttttctcacccgttggccaatttcctacatgagaaagaaagccctacagcaagtcgctgaattcttgaaatacgaagacgagaattctcaatacatctgcattggtgctgcacagaaggcactgtccatgttgtgctgctggatcgaaaaatccaactcagatgcgttcaagcgacatttggccagagtcgctgatttcctatggatcggtgaagatggcatgaaagtgcgggtctgtgctggtcagctatgggatgttgctttcgcagtccaagcaatactagcatgtaacatcgcagaagaatacagaggcaccctcaagaaagcgcacgatttcataaaggcctcccagatcatggacaatccttctggtgacttcagcagaaagtaccgccacatatctaagggagggtggggcttccaggttgcagatcaggggtggcaggtttcagattgcacagcagaagctctcaaggttttgttaatgctgtccagattttcatcagacattggaagcgatcagatggaaacatgccgcctatatgatgcagtgaatgtgttgttatccttacagaacccaaacggtggctatggaacttgggagctagctcgcacatatccatggatagagattttcaacatgacagagatatacgcagacatcatggtggagcatcagtacgtcgagtgtacctcatcagtcatgcaagcattagtattgtttcgggagaaggacccttggcatcgaaaagatgaaatagaccaatgcattaggggagctactgaattcatcgagaagttgcagaatgatgatggctcatggtttggatcttggggtatttgtttcacatatggcacatggtttgctattgagggtctatcagctgttgggcagagttatggtaacagcacttgtatccagaaggcctgcaagtttctcttagcaaagcagctaaagaatggtggatggggtgagagtcatctttcatctacaaccaaggcatacacaaatctggataaggagaagtcacacatagtgaacactgcgtgggcaatgttggcgctcatgaaagctggacaggcggaaagagatcccactccacttcacaaagctgcacggcttatcatgagtatgcagctcagcaatggcgacttcccacaggaggaaatgatcggaagtttcttgaaaaatggccccttgtgttacatggcttatcgcaacatatttcctatatgggcgcttggagagtatcagaaattagtcctccagtgtgacctccaatggccaaccttctag

SEQ ID NO: 22 - protein, 762 aa, Sorghum bicolor
  Sorghum bicolor triterpene synthase SbOSC1
MWRLKIAKGGPWLKSGNSHIGRETWEFDQDFGSKEEREAVDSAREEFKKNRFQMRHSSDILARMQLAKENGFSLDLQKTKDESPLVINSSTVSEILRKALNYFSAIQAHDGHWPGDFPGPLFTTATMIIVLYVTESLSSTLSSEHCKEICRYLYNRQNMDGGWGLHAEGESSMLSTALNYTTLRLLGENIDDGPDMSMLKARKWIHDHGGATMIPILGKVWLSVLGVFEWSGVNPIPPELFLLPSWVPIQPGRLWSHFRMAFIPMCYLYGKKFVGPITRLVMSLREELHIHPYKKIDWKQARKLCAKEDVYNPHTWLQECLSDCLYSFGEPFLTRWPISYMRKKALKQVAEFLKYEDENSQYICIGAAQKALSMLCCWIEKSNSDAFKRHLARVADFLWIGEDGMKVRVCAGQLWDVAFAVQAILACNIAEEYRSTLKKAHDFIKASQIMDNPSGDFSRKYRHISKGGWGFQVADQGWQVSDCTAEALKVLLMLSKFSSDIGSDQMETCRLYNAVNVLLSLQNPNGGYGTWELARTYPWMEIFNMTEIYADIMVEHQYVECTSSVMQALALFQEKYPWHRKDEIDQCIRGATEFIEKLQNDDGSWFGSWGICFTYGTWFAIEGLSAVGQSYGNSTCIRKACKFLLTKQLNNGGWGESYLSSRTKAYTNLDRQKSHIVNTAWAMLALMKAGQVERDPTPLHKAARLIMSMQLGNGDFPQEEMIGSFLKNGPLCYMAYRNIFPIWALGEYQKLVLQCDLQRPTF

SEQ ID NO: 23 - protein, 784 aa, Oryza sativa
  rice Oryza sativa strain OsOSC8 triterpene synthase
MWKLKIAEGGPWLKSGNSHVGRETWEFDPNFGTSEEREAVEAARIEFQKNRFRTRHTSDVLARMQLAKANNFSIDLQKEKDGNPINIDTATVSDILKKALSYFSAIQAYDGHWPGDFPGPLFTTATMIIVLYVTESLTITLSSEHHKEICRYLYNRQNIDGGWGLHAEGESSMLSTALNYTALRLLGENVDDGPDISMHKARKWIHDHGGATMIPILGKVWLSVLGVFDWSGVNPIPPELFLLPSFVPIQPGRLWSHFRMAFIPMSYLYGKKFVGPITRLVISLREELHIHPYKKIDWKEARKLCAKEDAYNPHMWLQECLSDCLYSFGEPFLTRWPISYMRKRALYQIAEFLKYEDENSQYICIGAAQKALSMLCCWIENPNSDAFKRHLARVADFLWVGEDGMKVRVCAGQLWDVAFAVQAILACSIAEEFGSTLKKAHGFIKTSQIMDNPSGDFSRKYRHISKGGWAFQVADQGWQVSDCTAEALKALLLLSKCLSDGADYQMETYCYFDAVNVLLSLQNPNGGYGAWELARTYPWMEIFNMTEIYADIIVEHQYVECTSSVIQALALFREKYPGHRKDEIDQCIRKATEFIEKLQNDDGSWFGSWGICFTYGTWFAIEGLSAVGQCYDDSTCIRKACKFLLSKQLTNGGWGESHLSSRTKAYTNLDGEKSHIVNTAWAMLALMKAGQVERDPAPLHKAARLIMSMQLSDGDFPQEEMIGSFLKNGPLCYMAYRNIFPIWALGEYQKLVFQNYQTSSIKQTNIAPSAGNAALKNSASTTAP

SEQ ID NO: 24 - protein, 784 aa, Hordeum vulgare; identical to SEQ ID NO: 23
  barley Hordeum vulgare strain Varda triterpene synthase HvOSC1

(19) United States
(12) Patent Application Publication
(10) Pub. No.: US 2015/0020237 A1
(43) Pub. Date: Jan. 15, 2015

(54) Method for Preparing Fertility-Lowered Plant

(71) Applicant: Institute of Botany of the Chinese Academy of Sciences, Beijing (CN)

(72) Inventors: Xiaoquan Qi, Beijing (CN); Zheyong Xue, Beijing (CN); Yingchun Zhang, Beijing (CN); Xia Xu, Beijing (CN); Dan Liu, Beijing (CN)

(73) Assignee: Institute of Botany of the Chinese Academy of Sciences, Beijing (CN)

(21) Appl. No.: 14/362,900

(22) PCT Filed: Nov. 14, 2012
(86) PCT No.: PCT/CN2012/001546

§ 371 Date: Jun. 4, 2014

(30) Foreign Application Priority Data
Dec. 8, 2011 (CN) 201110406266.2

(51) Int. Cl. C12N 15/82; C12N 15/113

(52) U.S. Cl. CPC C12N 15/8287; C12N 15/113; C12N 2310/14
USPC 800/285; 435/320.1; 435/252.3; 800/298

(57) ABSTRACT

The present invention relates to a method for preparing fertility-lowered plant. The present invention provides an RNA interference vector and a method for obtaining a transgenic plant by introducing said RNA interference vector into a target plant; said transgenic plant is the following 1) or 2): 1) Sterile transgenic plant or 2) The fertility of said transgenic plant is lower than said target plant; the present invention also provides a method for cultivating a target plant to a sterile mutant or fertility-lowered mutant by sodium azide mutagenesis. The experiments of the present invention proved that the present invention provides various methods for preparing sterile lines or fertility-lowered lines; including RNA interference or TILLING (Targeting Induced Local Lesions IN Genomes) technology selection; sterile lines prepared by the methods of the present invention establish the basis of rice heterosis and crossbreeding.

### TECHNICAL FIELD

The present invention relates to biotechnology, especially to a method for preparing a fertility-lowered plant.

### BACKGROUND

Male sterility in plant is a botanical characteristic closely related to agricultural production and is the result of interaction between gene expression and environment during plant development. Autologous male sterility in plant can be used in breeding research including development and utilization of crop heterosis, conducting recurrent selection and backcross, etc. as a genetic tool without artificial emasculation. It provides the possibility to produce a large amount of hybrid seeds by utilizing plant male sterility to breed various male sterile lines and then producing hybrid seeds in large quantities by means of genetic engineering so that heterosis of many crops especially self-pollinated crops can be utilized in production.

Rice is an important cereal crop in China. The discoveries of rice heterosis and hybrid breeding methods make great contribution to rice production, increasing the production by more than 20% compared with conventional breeding. Following the discovery of Environment-sensitive Genetic Male-sterile Rice, national academy member YUAN Long-ping put forward a two-line breeding method which makes the strategy of rice hybrid breeding simple and convenient, improves the quality in many aspects and increases the production by 5-10%. Nongken 58S is the first photoperiod-sensitive male sterile line which as well as its transformed japonica rice sterile line and indica rice sterile line can be induced to be sterile by long day and fertile by short day. Meanwhile, they are also called Photo thermo sensitive genetic male sterile line because their fertility is affected by temperature. By means of gene mapping, it is found that the Photo thermo sensitive genetic male sterility is regulated by a noncoding precursor RNA. However, abnormal low temperature (<23° C.) in the summer will lead to failure in production of hybrid seeds, so the use of photo thermo sensitive genetic male sterile lines are affected to some extent.

Besides the influence of light and temperature, humidity is also an important environment factor relating to plant growth and development. To date, there isn't any report on the relationship between humidity and rice male sterility. However, a male sterile mutant pop1 which is related to humidity was found in Arabidopsis thaliana as early as 1993. The pollen of the mutant cannot absorb water from the stigma due to the lack of long chain fatty acid and wax in the tryphine of the pollen surface, thereby leading to the failure of pollination and sterility (but the fertility of the mutant was restored by transferring the mutant from environment with a relative humidity of 70% to a high humidity box with a relative humidity of 90%. Preuss et al., Gene Dev. 1993, 7:947-985).

### SUMMARY

In one aspect, the present invention provides an RNA interference vector.

The RNA interference vector of the invention is obtained by inserting the DNA molecule as shown in SEQ ID NO.1 into a pH7GWIWG2(II) vector.

The above RNA interference vector is obtained by inserting the DNA molecule as shown in SEQ ID NO.1 into a pH7GWIWG2(II)vector by means of homologous recombination. In particular, the vector is obtained by inserting the DNA molecule as shown in SEQ ID NO.1 into a pH7GWIWG2(II)vector in a forward direction and reverse direction by means of homologous recombination.

The above RNA interference vector is prepared according to a method comprising the following steps:

1) Obtaining an intermediate vector with the DNA molecule as shown in SEQ ID NO.1 and a pDONR221 vector by BP reaction;

2) Obtaining an RNA interference vector with said intermediate vector and a pH7GWIWG2(II) vector by LR reaction.

The aforementioned DNA molecule as shown in SEQ ID NO.1 is prepared according to the following method: PCR amplification of rice cDNAs with primer pair A, and the obtained PCR product is the DNA molecule as shown in SEQ ID NO.1.

Said primer pair A consists of the single chain DNAs as shown in SEQ ID NO.3 and SEQ ID NO.4.

Recombinant bacteria or a transgenic cell line comprising said RNA interference vector also fall into the protection scope of the present invention.

Use of said RNA interference vector, recombinant bacteria or transgenic cell line in cultivating rice sterile lines or reducing rice fertility also falls in the protection scope of the present invention.

The second aspect of the present invention is to provide a method for cultivating a transgenic plant.

The method provided by the present invention is directed to obtain the transgenic plant by introducing said RNA interference vector into a target plant; wherein said transgenic plant comprises the following 1) or 2):

1) Sterile transgenic plant; or 2) The fertility of said transgenic plant is lower than said target plant;

wherein said target plant is specifically a monocot plant; said monocot plant is specifically rice.

The transgenic plant obtained by said method is also within the protection scope of the present invention; Said transgenic plant is a sterile transgenic plant or fertility-lowered transgenic plant; wherein said plant is specifically a monocot plant; said monocot plant is further specifically rice. Said fertility-lowered transgenic plant is a transgenic plant whose fertility is lower than the target plant.

The third aspect of the present invention is directed to provide a method for cultivating a target plant into a sterile mutant or fertility-lowered mutant.

The method provided by the present invention comprises the following steps:

1) Mutating seeds of a target plant; Using primer pair B and fluorescently labeled primer pair B to specifically amplify genes encoding triterpene synthase from a target plant;

wherein mutating the seeds of a target plant is performed by treating a number of seeds of the target plant with sodium azide, thereby obtaining the mutated seeds;

wherein said primer pair B is used to specifically amplify the triterpene synthase in the target plant is designed according to the particular sequences of triterpene synthase gene in the target plant; wherein each primer of said fluorescently labeled primer pair B is labeled with different fluorescently labeled probes; wherein said different fluorescently labeled probes have different wavelengths;

2) Cultivating said mutagenized seeds to obtain the first generation of mutation M1; Self-cross of the first generation of mutation M1 was carried out to obtain the second generation of mutation M2;

3) Extracting genomic DNA of individual plant of the second generation of mutation M2; Mixing genomic DNA of individual plants of M2 (with number n) to obtain a DNA pool; wherein n is 2-8;

4) Using each said DNA pool as a template and both said primer pair B and fluorescently labeled primer pair B to perform amplification, so as to obtain PCR products;

5) Digesting said PCR products with endonuclease CELI to obtain enzyme-digested products of the DNA pool;

6) detecting the enzyme-digested products of each said DNA pool by electrophoresis; wherein if enzyme-digested products of said DNA pool generate bright dots under all wavelengths of different fluorescently labeled probes, individual plants of M2 generation (with number n) represented by said DNA pool contain or may contain fertility-lowered mutants or sterile mutants; wherein if the enzyme-digested products of said DNA pool do not generate bright dots under all wavelengths of different fluorescently labeled probes, M2 generation (number n) represented by said DNA pool do not contain or may not contain fertility-lowered mutants or sterile mutants; wherein said fertility-lowered mutants are plants whose fertility is lower than that of said target plant.

Following said step 6), said method further comprises the following steps:

Genomic DNA of individual plant of M2 (with number n) which contain or may contain fertility-lowered mutants or sterile mutants is mixed with genomic DNA of said target plant; steps 4)-5) are repeated to obtain enzyme-digested products of individual plant of M2.

Each of said enzyme-digested products of individual plant of M2 is detected by electrophoresis; wherein if said enzyme-digested products of individual plant of M2 generate bright dots under all wavelengths of different fluorescently labeled probes, said individual plant of M2 is or may be a sterile mutant or fertility-lowered mutant; wherein if said enzyme-digested products of individual plant of M2 do not generate bright dot under all wavelengths of different fluorescently labeled probes, said individual plant of M2 is not or may not be a sterile mutant or fertility-lowered mutant.

In step 1) of said method, treating a number of seeds of the target plant with sodium azide comprises immersing a number of seeds of the target plant in an aqueous solution of sodium azide with a concentration of 2 mM for 6 hours under room temperature.

The amino acid sequence of said triterpene synthase is shown in SEQ ID NO. 2;

Wherein said different fluorescently labeled probes refer to fluorescently labeled probe DY-682 with a wavelength of 682 nm and fluorescently labeled probe DY-782 with a wavelength of 782 nm;

The nucleotide sequence of the gene encoding said triterpene synthase is shown in SEQ ID NO. 5;

The primer pair B is one of the following primer pairs represented by 1)-3):

1) Primer pair consisting of the single chain DNA molecule as shown in SEQ ID NO. 6 and the single chain DNA molecule as shown in SEQ ID NO. 7;

2) Primer pair consisting of the single chain DNA molecule as shown in SEQ ID NO. 8 and the single chain DNA molecule as shown in SEQ ID NO. 9;

3) Primer pair consisting of the single chain DNA molecule as shown in SEQ ID NO. 10 and the single chain DNA molecule as shown in SEQ ID NO. 11;

wherein said target plant is a monocot plant; wherein said monocot plant is a monocot graminaceous plant; specifically, said monocot graminaceous plant is rice.

The above fertility-lowered mutant is a mutant whose fertility is lower than the target plant. In one embodiment, the target plant is specifically rice, and fertility-lowered mutant is a mutant whose fertility is lower than that of target rice.

Sterile mutant or fertility-lowered mutants prepared by the above method are also within the protection scope of the present invention.

The Deposit Number of the above-mentioned fertility-lowered mutant is CGMCC NO. 6150.

Use of the above transgenic plants or the above sterile mutant or fertility-lowered mutant in the production of hybrid seeds is also within the scope of protection of the present invention.

The above fertility-lowered mutant is a mutant whose fertility is lower than that of a target plant; said target plant is a monocot plant; said monocot plant is a monocot graminaceous plant; said monocot graminaceous plant is specifically rice; In one embodiment, a fertility-lowered mutant is specifically a mutant whose fertility is lower than that of target rice.

The fourth aspect of the present invention is to provide a method for obtaining a sterile mutant or fertility-lowered mutant.

The method provided by the present invention is to obtain a sterile mutant or fertility-lowered mutant by silencing or inactivating the gene encoding triterpene synthase in a target plant; said fertility-lowered mutant is a plant whose fertility is lower than that of the target plant.

In the above method, said target plant is a monocot or dicot plant; said monocot plant is specifically a monocot graminaceous plant;

said monocot graminaceous plant is rice, wheat, barley, sorghum or maize;

The amino acid sequence of the triterpene synthase of rice is shown in SEQ ID NO. 2; the nucleotide sequence of the gene encoding triterpene synthase of rice is shown in SEQ ID NO. 5.

The amino acid sequence of the triterpene synthase of wheat is shown in SEQ ID NO. 15; the nucleotide sequence of the gene encoding triterpene synthase of wheat is shown in SEQ ID NO. 14.

The amino acid sequence of the triterpene synthase of barley is shown in SEQ ID NO. 17; the nucleotide sequence of the gene encoding triterpene synthase of barley is shown in SEQ ID NO. 16.

The amino acid sequence of the triterpene synthase of sorghum is shown in SEQ ID NO. 18; the nucleotide sequence of the gene encoding triterpene synthase of sorghum is shown in SEQ ID NO. 19.

The amino acid sequence of the triterpene synthase of maize is shown in SEQ ID NO. 20; the nucleotide sequence of the gene encoding triterpene synthase of maize is shown in SEQ ID NO. 21.

The method of the aforementioned silencing or inactivation of the gene encoding triterpene synthase in a target plant can be specifically RNA interference of expression of the gene encoding triterpene synthase in a target plant or point mutation of the gene encoding triterpene synthase in a target plant.

In the above method, said silencing or inactivating of the gene encoding triterpene synthase in rice can be at least one of the following 1)-3):

1) The nucleotide residue at the 764 position of the gene encoding triterpene synthase in rice is mutated from G to A;

2) The nucleotide residue at the 809 position of the gene encoding triterpene synthase in rice is mutated from G to A;

3) The nucleotide residue at the 1431 position of the gene encoding triterpene synthase in rice is mutated from G to A.

The fifth aspect of the present invention is to provide a method for restoring or improving fertility of an original plant.

The method provided by the present invention comprises the following steps: maintaining the humidity for growth of plant inflorescence at 80-100% during anthesis of an original plant; wherein said original plant is a sterile mutant or fertility-lowered mutant.

In the above method, said sterile mutant or fertility-lowered mutant is the aforementioned transgenic plant or the aforementioned sterile mutant or fertility-lowered mutant.

In the above method, the time period of said maintaining the humidity for growth of plant inflorescence is one week;

the method for maintaining the humidity for growth of plant inflorescence comprises wrapping the whole inflorescence of said original plant;

said wrapping comprises specifically using a plastic bag to slip over the whole inflorescence or using preservative film to cover the whole inflorescence.

The fertility-lowered mutant named P34E8 selected above is a mutant strain of OsOSC8 (mutant S6) which has been deposited in China General Microbiological Culture Collection Center (CGMCC for short, address: No. 3, Courtyard No. 1, West Road Beichen, Chaoyang District, Beijing) on 28 May 2012 with a deposit Number of CGMCC No. 6150; the classification and nomenclature is rice (Oryza sativa).

Unless particularly noted or separately defined, the terms of science and technology used in this text have the unambiguously same meaning as is well known to the person skilled in the art. Moreover, the material, method and embodiments of the text are intended to explain and elaborate the present invention rather than limitation or restriction.

### DESCRIPTION OF DRAWINGS

FIG. 1 shows result of western blot analysis of the protein of RNAi lines.

FIG. 2 shows statistical result of setting percentage under natural condition and setting percentage after moisturizing treatment.

FIG. 3 shows electrophoretogram of detected mutants.

FIG. 4 shows inflorescence (A, B), floret (C, D, bars=0.5 cm), I₂—KI staining (E, F, bars=100 μm) and Alexander staining (G, H, bars=100 μm) of wild type (WT) and mutant (P34E8).

FIG. 5 shows germination of pollens of wild type (WT) and mutant (P34E8) in culture medium, bars=100 μm.

FIG. 6 shows adhesion and germination of pollens of wild type (WT) and mutant (P34E8) on stigmas at different times, bars=100 μm.

FIG. 7 shows adhesion and germination of pollens on stigmas after reciprocal cross is made between wild type (WT) and mutant (P34E8), bars=100 μm.

FIG. 8 shows fructification of inflorescence of wild type (WT), mutant (P34E8) and homozygous mutant after moisturizing treatment, bars=2 cm.

FIG. 9 shows electrophoretogram of amplified fragments of homologous genes in barley and wheat.

FIG. 10 shows sequence alignment of homologous proteins of OsOSC8 in graminaceous crops and possible effective mutant sites.

### EMBODIMENTS

Unless specially illustrated, the experimental methods used in the following embodiments are all conventional methods.

Unless specially illustrated, all of the materials and reagents, etc. used in the following embodiments can be obtained from commercial sources.

All of the quantitative tests in the following embodiments are repeated three times, and the results are mean value or mean value±standard deviation.

The amino acid sequence of triterpene synthase OsOSC8 is shown in SEQ ID NO.2; the nucleotide sequence encoding the gene is shown in SEQ ID NO.5.

### Example 1

### Preparation of Fertility-Lowered Transgenic Rice by RNA Interference

I Obtaining of RNA Interference Vector of OsOSC8

1. Obtaining of Gateway Intermediate Vector pDONR221/osc8-1.

The primers were designed according to the gene sequence of OsOSC8: Sequence attB1 was added to 5′ end of the sense primer and sequence attB2 was added to 5′ end of the antisense primer by using Gateway technology of Invitrogen, USA.

Primer pair 1:
sense:
(SEQ ID NO. 3)
5′-AAAAAGCAGGCTGGCTGCACGGATAGAGTT-3′
antisense:
(SEQ ID NO. 4)
5′-AGAAAGCTGGGTGCCTGTATGGCTGAGAAA-3′

Total RNA was extracted from rice Zhonghua 11 (Orazy sativa L. ssp japonica; See, Zhong-Hai Ren et al., 2005, A rice quantitative trait locus for salt tolerance encodes a sodium transporter. Nature Genetics 37, 1141-1146; the public can obtain the material from Institute of Botany, the Chinese Academy of Sciences) and reverse transcribed to obtain cDNA.

PCR amplification of the obtained cDNA with primer pair 1, and the resultant fragment 1 has a size of about 200 bp; Sequencing results confirmed the nucleotide sequence of fragment 1 was the sequence as shown in SEQ ID NO.1; The PCR reaction mixture comprises 2 pmol of each primer of primer pair 1, 10 μl of PCR Mix (Genestar A112-01), 2 μl of cDNA, adding double-distilled water to 20 μl. The PCR protocol was: Denaturation at 94° C. for 3 minutes, followed by 30 cycles of denaturation at 94° C. for 30 seconds, anneal at 55° C. for 30 seconds, and extension at 72° C. for 30 seconds, and final extension at 72° C. for 10 minutes.

The obtained fragment 1 was mixed with equal molar of pDONR221 vectors (Invitrogen 12535-037) and incubated at 25° C. for 1 hour, i.e. BP reaction (Invitrogen 11789-020) was carried out to generate an entry vector. The entry vector was transformed into Escherichia coli DH5α by heat shock. The transformed cells were spread on a LB medium plate plus 50 mg/L of kanamycin for overnight to obtain transformants (simple principle: original plasimid pDONR221 comprises a lethal gene ccd B, so transformants can't survive; only when ccd B gene is replaced by exogenous fragment, bacterial colonies can survive).

| | | | |
|---|---|---|---|
| BP reaction system: | Fragment 1/Fragment 2 | 100 ng/μl | 1 μl |
| | pDONR/Zeo vector | 100 ng/μl | 1 μl |
| | BP Clonase II Enzyme mix | | 2 μl |
| | TE Buffer (pH = 8.0) | | 6 μl |

BP reaction procedure: incubation at 25° C. for 1 hour, then 1 μl of protease K was added and mixed, followed by incubation at 37° C. for 10 minutes.

Colony PCR of the transformants was carried out using Primer pair 1. Single colonies which could obtain PCR product of 200 bp were preserved as positive clone.

Plasmid was extracted from positive clone and then sequenced. Plasmids extracted from the confirmed positive clone represents the vector obtained by inserting the sequence as shown in SEQ ID NO.1 into pDONR221 vector and was named pDONR/osc8-1.

2. Obtaining Gateway Terminal Vector pH7GWIWGII-osc8-1 (i.e., RNA Interference Vector of OsOSC8).

The pDONR/osc8-1 vectors obtained by the above mentioned step 1 were mixed with equal molar of pH7GWIWG2(II) vectors (Damme et al., Somatic Cytokinesis and Pollen Maturation in Arabidopsis Depend on TPLATE, Which Has Domains Similar to Coat Proteins. Plant Cell, 2006, 18: 3502-3518. The public can obtain the material from Institute of Botany, the Chinese Academy of Sciences) and incubated at 25° C. for 1 hour, i.e., LR reaction (Invitrigen 11791-020). The mixture was transformed into Escherichia coli DH5α by heat shock and the transformed cells were spreaded on a plate plus 100 mg/L of spectinomycin. Incubate at 37° C. overnight to obtain respective transformants (the principle is the same as BP reaction).

| | | | |
|---|---|---|---|
| LR reaction system: | pH7GWIWG2 (II) | 100 ng/μl | 1 μl |
| | pDONR/osc8-1 | 100 ng/μl | 1 μl |
| | LR Clonase Reaction Buffer | | 4 μl |
| | TE Buffer (PH = 8.0) | | 10 μl |
| | LR Clonase enzyme mix | | 4 μl |
| | | | 20 μl |

LR reaction procedure: incubation at 25° C. for 1 hour, then 2 μl of protease K was added and mixed followed by incubation at 37° C. for 10 minutes.

Plasmid was extracted from single colonies of transformants and then sequenced. Plasmid in confirmed single clone of transformants was named pH7GWIWGII-osc8-1 and contained the sequence as shown in SEQ ID NO.1 which was inserted into the pH7GWIWG2(II) vector in forward direction and reverse directions. It was an RNA interference vector.

II Obtaining Fertility-Lowered Plants by Using RNA Interference Vector of OsOSC8

1. Obtaining Recombinant Agrobacterium tumefaciens

The RNA interference vector pH7GWIWGII-osc8-1 obtained by the above step I was introduced into Agrobacterium tumefaciens strain EHA105 (K L Piers et al., 1996, Agrobacterium tumefaciens-mediated transformation of yeast. PNAS February 20, vol. 93 no. 4 1613-1618; the public can obtain the material from Institute of Botany, the Chinese Academy of Sciences) by means of electroporation under a voltage of 1800V to obtain transformants.

The resultant mixture was cultured at 28° C. for two days and then single clone were picked up to perform PCR amplification (using primer pair 1). Colonies with the expected fragments of 200 bp are positive clone named EHA105/pH7GWIWGII-osc8-1 and preserved in 15% of glycerol at −80° C.

2. Obtaining RNA Interference Transgenic Plants

1) Cultivation of rice calli

a) Sterilized ddH₂O, 70% ethanol, 1% corrosive sublimate and sterilized 100 ml-triangular flasks were placed in hood and then shut the hood with glass window. Ultraviolet radiation was performed after 30 minutes.

b) Seeds of rice Zhonghua 11 (hereinafter refers to wild-type rice) were put into a sterilized triangular flask and rinsed by sterilized ddH₂O for 3 times to remove floating seeds and impurities on the surface of the seeds.

c) Following discard of ddH₂O, the seeds were sterilized by 70% ethanol for 8 minutes. The triangular flask was shaked from time to time to facilitate a complete sterilization.

d) Following discard of 70% ethanol, the seeds were sterilized by 1% corrosive sublimate for 8 minutes. Do not use too much corrosive sublimate, only submerging the surface of the seeds.

e) Following discard of 1% corrosive sublimate, the seeds were rinsed by sterilized ddH₂O for 4 times and then added appropriate amount of sterilized ddH₂O (liquid level is 1 cm higher than the surface of the seeds) followed by sealing with a sealing film and immersion for 12 hours.

f) Mature embryos of rice seeds were cut off under sterile conditions and inoculate in induction medium NB2 and cultured in dark at 25° C. for 3-4 weeks.

g) Calli growing from mature embryos were cut off and transferred to subculture medium NB 1 and cultured in dark at 25° C. for 2 weeks.

h) Calli in good condition were cut into pieces of callus with a size of a mung bean and transferred to NB 1 medium and cultured in dark at 25° C. for 4 days.

2) Preparation and transformation of Agrobacterium tumefaciens a) EHA105/pH7GWIWGII-osc8-1 was inoculated in YEB+RIF+SPE liquid medium (i.e. YEB medium containing 25 mg/L of rifampicin and 100 mg/L of spectinomycin) in a proportion of 1:100 and cultured at 28° C., 230 rpm for 23 hours.

b) The cultured bacterial suspension was then inoculated in YEB+RIF+SPE liquid medium in a proportion of 1:50 and cultured at 28° C., 230 rpm till reach an OD600 of 0.5. The bacterial suspension was collected into a 50 ml-sterilized centrifuge tube and precipitated by centrifugation at 4000 g for 5 minutes. The supernatant was discarded.

c) Collected bacteria were resuspended with 50 ml AAM-AS medium in a sterilized 100 ml-triangular flask and shaked in a shaker for 45 minutes to make the bacteria uniformly dispersed in medium.

d) Calli obtained by the above 1) were precultured for 4 days and then immersed in resuspended bacterial suspension for 5-10 minutes. The triangular flask was shaked occasionally.

e) Discard the bacterial suspension. Calli were placed on sterile filter papers and transferred to co-culture medium NB2C (a layer of sterile filter paper with appropriate size was placed on the surface of the medium in advance) after bacterial suspension was absorbed by filter papers. Calli were cultured in dark at 25° C. for 4 days to obtain calli which were co-cultured for 4 days.

3) Selection and regeneration of positive calli

a) Calli which were co-cultured for 4 days in the above 2) were transferred to NB1 medium containing hygromycin (20 mg/L) and Timentin (225 mg/L) and cultured in dark at 25° C. for 2 weeks to select positive transformants.

b) Calli infected with bacteria in the first selection were discarded and the rest were transferred to NB1 medium containing hygromycin (20 mg/L) and Timentin (180 mg/L) and cultured in dark at 25° C. for 2 weeks to perform the second selection.

c) Calli infected with bacteria in the second selection were discarded and the rest were transferred to NB1 medium containing hygromycin (50 mg/L) and Timentin (180 mg/L) and cultured in dark at 25° C. for 2 weeks to perform the third selection.

d) When calli grew to 0.5-0.8 cm, check them under fluorescence microscope. Select calli with green fluorescence. The selected calli were transferred to regeneration medium DR1 and cultured in dark for 1 week followed by culture in light (at 23° C. under a 12/12 h (day/night) photoperiod with light supplied at an intensity of 5000 lux) for 1 week.

e) Regenerated seedlings or calli were transferred to regeneration medium DR2 all together and cultured in light (the same as above) for 2 weeks.

f) Regenerated seedlings were transferred to grass carbon soil when seedlings grew to about 8 cm and cultivated at 28° C. in glass green house. 30 RNA interference transgenic rice plants of T0 generation were obtained.

pDONR 221 vectors and pH7GWIWG2(II) vectors were mixed for LR reaction using the same method to obtain RNA interference blank vector. Then RNA interference blank vectors were transformed into wild-type rice by Agrobacterium tumefaciens to obtain blank vector transgenic rice RNAi-CK-3.

Part of the above used medium are as follows in tables 1-4:

TABLE 1
Formula of medium

| Components | NB1 (1 L) | NB2 (1 L) | NB2C (1 L) |
|---|---|---|---|
| N6 major element (10×) | 100 ml | 100 ml | 100 ml |
| Fe salt (100×) | 10 ml | 10 ml | 10 ml |
| inositol (100×) | 10 ml | 10 ml | 10 ml |
| B5 organics (100×) | 10 ml | 10 ml | 10 ml |
| B5 microelement (1000×) | 1 ml | 1 ml | 1 ml |
| 2, 4-D (1 mg/ml) | 0.5 | 2 ml | 2 ml |
| L-Glutamine | 0.5 g | 0.5 g | 0.5 g |
| L-Proline | 0.5 g | 0.5 g | 0.5 g |
| CH (acid hydrolysis of | 0.3 g | 0.3 g | 0.3 g |
| casein) | | | |
| Sucrose | 30 g | 30 g | 30 g |
| Glucose | | | 10 g |
| AS (1 mg/ml) | | | 2 ml |
| Adjust pH value to | 5.8 | 5.8 | 5.2 |

TABLE 2
Formula of AAM-AS medium

| AAM-AS major element | | AAM-AS amino acid | | AAM-AS vitamin | |
|---|---|---|---|---|---|
| (10×) 1 L | | (100×) 100 ml | | (1000×) 100 ml | |
| CaCl₂•2H₂O | 1.5 g | Glutamine | 8.76 g | Glycine | 0.75 g |
| KH₂PO₄ | 1.2 g | Aspartic acid | 2.66 g | Thiamine hydrochloride | 0.01 g |
| MgSO₄•7H₂O | 2.5 g | Arginine | 1.74 g | Pyridoxine hydrochloride | 0.05 g |

| | | | | |
|---|---|---|---|---|
| KCl | 29.5 g | (dissolve separately and | nicotinic acid | 0.05 g |
| | | then mix) | | |

| Components | DR1 (1 L) | DR2 (1 L) | AAM-AS (1 L) |
|---|---|---|---|
| MS major | 100 ml | 100 ml | AAM-AS major element (10×) |
| element (10×) | | | 100 ml |
| Fe salt (100×) | 10 ml | 10 ml | AAM-AS amio acid (100×) |
| | | | 10 ml |
| inositol (100×) | 10 ml | 10 ml | inositol (100×) |
| | | | 10 ml |
| MS organics | 10 ml | 10 ml | AAM-AS vitamin (1000×) |
| (100×) | | | 1 ml |
| MS microelement | 1 ml | 1 ml | B5 microelement (1000×) |
| (1000×) | | | 1 ml |
| 6-BA (1 mg/ml) | 1 ml | 1 ml | CH (acid hydrolysis of casein) |
| | | | 0.5 g |
| KT (1 mg/ml) | 500 μl | 500 μl | Sucrose |
| | | | 30 g |
| NAA (1 mg/ml) | 250 μl | 500 μl | 2,4-D (1 mg/ml) |
| | | | 500 μl |
| ZT (0.2 mg/ml) | 1 ml | 1 ml | AS (50 mg/ml) |
| | | | 1 ml |
| CH (acid | 0.3 | 0.3 | |
| hydrolysis of | | | |
| casein) | | | |
| Sucrose | 30 | 30 | |
| Sorbitol | 30 | 30 | |
| Adjust pH value | 5.8 | 5.8 | 5.2 |
| to | | | |

TABLE 3
Formula of major element
major element (10×) 1 L

| Components | N6 major element | MS major element |
|---|---|---|
| CaCl₂•2H₂O | 1.66 g | 4.4 g |
| NH₄NO₃ | | 16.5 g |
| KNO₃ | 23.8 g | 19 g |
| (NH₄)₂SO₄ | 4.63 g | |
| KH₂PO₄ | 4 g | 1.7 g |
| MgSO₄•7H₂O | 1.85 g | 3.7 g |

TABLE 4
Formula of microelement
Microelement (1000×) 1 L

| Components | B5 microelement | MS microelement |
|---|---|---|
| MnSO₄•4H₂O | 10 g | 22.3 g |
| ZnSO₄•7H₂O | 2 g | 8.6 g |
| H₃BO₃ | 3 g | 6.2 g |
| KI | 0.75 g | 0.83 g |
| Na₂MoO4•2H₂O | 0.25 g | 0.25 g |
| CuSO₄•5H₂O | 0.025 g | 0.025 g |
| CoCl₂•6H₂O | 0.025 g | 0.025 g |

TABLE 5
Formula of organics
Organics (100×) 200 ml

| Components | B5 organics | MS organics |
|---|---|---|
| Glycine | | 0.04 g |
| Thiamine | 0.2 g | 0.008 g |
| hydrochloride | | |
| Pyridoxine | 0.02 g | 0.01 g |
| hydrochloride | | |
| nicotinic acid | 0.02 g | 0.01 g |

TABLE 6
Other formulae

| Fe salt (100×) 200 ml | | Inositol (100×) 200 ml |
|---|---|---|
| Liquid A | FeSO₄•7H₂O 100 ml | inositol 2 g |
| | (27.8 mg/ml) | ddH₂O 200 ml |
| Liquid B | Na-EDTA 100 ml | |
| | (37.3 mg/ml) | |

Mix A and B

3. Western Blot Analysis of RNA Interference Transgenic Rice

When RNA interference transgenic rice of the T0 generation obtained by the above 2 was at the booting stage, protein was extracted from young panicles at the vacuolated microspore stage and analyzed by western blot (antibody was polyclonal antibody of OsOSC8 protein which was isolated from serum of rabbits immunized with OsOSC8 protein. The antibody can be monoclonal antibody of OsOSC8 protein made by Shanghai Abmart Company). Measure the content of OsOSC8 protein.

The result is shown in FIG. 1, wherein Zh11 represents wild-type rice, RNAi-9, RNAi-12, RNAi-20 and RNAi-21 represent RNA interference transgenic rice of the TO generation and RNAi-CK-3 represents blank vector transgenic rice. It can be seen that expression of OsOSC8 in transgenic lines RNAi-9, RNAi-12 and RNAi-20 was reduced obviously, whereas expression of OsOSC8 in the control line RNAi-CK-3 is equal to wild-type rice.

The results show that RNAi-9, RNAi-12, RNAi-20 and RNAi-21 are positive RNA interference transgenic rice of the T0 generation which are mutants obtained by silencing of the OsOSC8 gene in rice by means of RNA interference.

4. Statistics of Setting Percentage of RNA Interference Transgenic Rice

Setting in natural condition: RNA interference transgenic rice of the T0 generation which were named as RNAi-9, RNAi-12, RNAi-20 and RNAi-21 and identified by the above 3 were cultivated in glass green house with natural lighting and maintained at 18° C./25° C. (night/day). Growth conditions: temperature 18° C./30° C. (night/day), humidity 30%-50%, natural lighting. Setting percentage of each panicle was analyzed statistically 4 weeks after flowering of RNA interference transgenic rice of the T0 generation and the results were compared to blank vector transgenic rice RNAi-CK-3 and wild-type rice ZH11. 5 panicles of each transgenic plant were analyzed and the results were mean value±standard deviation.

Setting percentage=the percentage of plump seeds in total seeds (plump seeds+empty seeds);

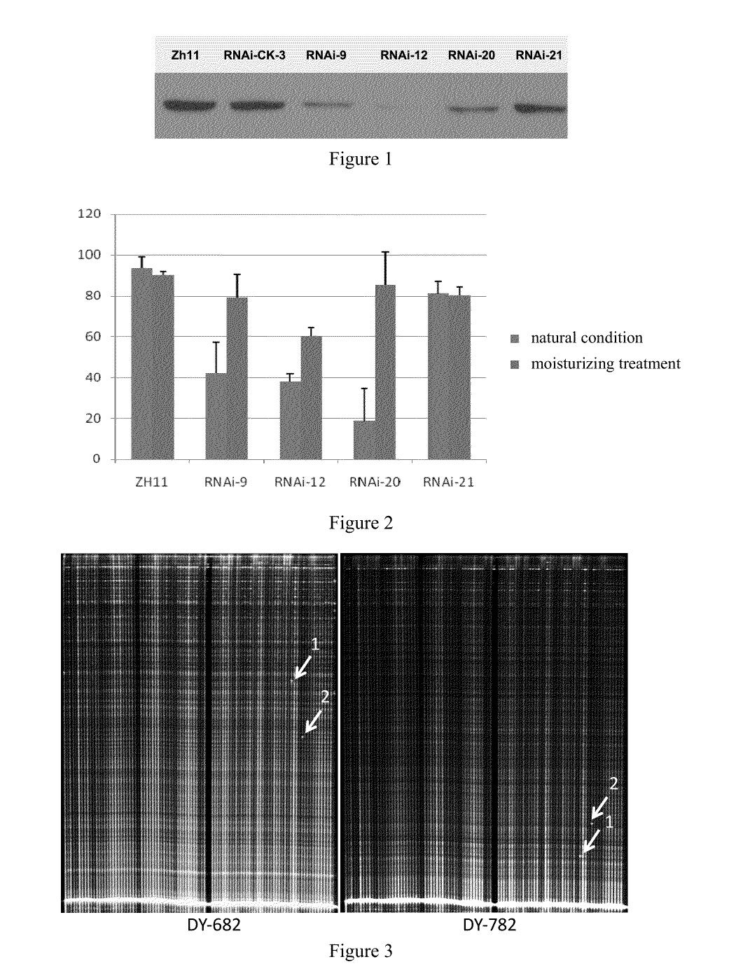

The results are shown in FIG. 2.

Setting in natural condition, the setting percentages of wild-type rice ZH11, RNA interference transgenic rice of the T0 generation RNAi-9, RNAi-12, RNAi-20 and RNAi-21 are 93.5±5.3%, 42.1±15.3%, 37.9±3.9%, 18.6±16.1% and 81.1±6.1% respectively.

There is no significant difference in setting percentage between blank vector transgenic rice RNAi-CK-3 and wild-type rice.

Thus the natural setting percentages of RNA interference transgenic rice of the T0 generation fall below 40%, wherein that of RNAi-20 fall below 20% and the natural setting percentage of wild-type rice is above 80%.

It indicates that fertility of RNA interference transgenic rice of the T0 generation obtained by silencing of the OsOSC8 gene in rice by means of RNA interference is lowered compared to wild-type rice. Sterile transgenic rice can be obtained by selecting more transgenic plants.

### Example 2

### Obtaining Fertility-Lowered Mutant by Utilizing TILLING Technology

I Selection of Fertility-Lowered Mutant by Utilizing TILLING Technology

1. Mutating Seeds of a Target Plant; Designing Primer Pairs for Specifically Amplifying Genes Encoding Triterpene Synthase in the Target Plant.

1) Mutating Seeds of a Target Plant

20,000 grains of seeds of Zhonghua 11 rice were immersed in 2 mM aqueous solution of sodium azide at 25° C. for 6 hours to obtain mutated seeds.

2) Primer Design

Primers were designed according to OsOSC8 gene sequence encoding triterpene synthase in rice. The primer can specifically amplify said gene. The sequences were:

Primer pair 2:
Sense primer OsOSC8T1F:
(SEQ ID NO. 6)
GAGGTCAAGTCGTCTTCTGCAATTA;
Antisense primer OsOSC8T1R:
(SEQ ID NO. 7)
ATTTGTCTGCGCTCTGCACATG;
Primer pair 3:
Sense primer OsOSC8T13F:
(SEQ ID NO. 8)
GCTTAAAGGTAAATTTCAGGCTTCC;
Antisense primer OsOSC8T13R:
(SEQ ID NO. 9)
CGATCAGAATCAATTAAACCCAGAC;
Primer pair 4:
Sense primer OsOSC8T17F:
(SEQ ID NO. 10)
TCATCCTTAGATTAATTAGCCGACA;
Antisense primer OsOSC8T17R:
(SEQ ID NO. 11)
CATAAGGATCTCATAAAATCGACCA;

Each of the above primer pairs were labeled with fluorescently labeled probes of different wavelengths. Fluorescently labeled probes having different wavelengths are fluorescent dye DY-682 with a wavelength of 682 nm (Eurofins DNA Campus Ebersberg, Germany) and fluorescent dye DY-782 with a wavelength of 782 nm (Eurofins DNA Campus Ebersberg, Germany).

The 5′ end of all sense primers were labeled by DY-682 fluorescence (DY-682), while the 5′ end of all antisense primers were labeled by DY-782 fluorescence (DY-782).

2. Cultivation

The above obtained mutated seeds were rinsed with water and cultivated in field to obtain the first generation of mutation M1. Self-cross of the first generation of mutation M1 to obtain the second generation of mutation M2. Then self-cross of the second generation of mutation M2. Harvest and preserve seeds of the third generation of mutation M3.

3. Extraction of DNA and Construction of Gene Pool

Seeds of the second generation of mutation M2 were harvested. Random 12 plants of each line of M2 were planted and genomic DNA of individual plant of the second generation of mutation M2 was extracted and preserved at −20° C. for subsequent use. Genomic DNAs from 4 individual plants of M2 were mixed (mixed with equal quantity) to obtain a DNA pool. Detect the quality and measure the concentration of DNA and then the DNA is uniformally mixed.

4. PCR Amplification

PCR amplification of the above DNA pools with primer respective pair 2, 3, and 4 to obtain 3 types of PCR amplified products. Procedure and system of PCR amplification were as follows:

TABLE 7
PCR system

| Master mix | 10 ul for each reaction | ×104 |
|---|---|---|
| dNTP (2.5 mM) | 0.8 μl | 83.2 μl |
| 10× Buffer | 1 μl | 104 μl |
| Sense primer with | 0.096 μl | 9.984 μl |
| fluorescence label (10 μM) | | |
| Sense primer without | 0.064 μl | 6.656 μl |
| fluorescence label (10 μM) | | |
| Antisense primer with | 0.128 μl | 13.312 μl |
| fluorescence label (10 μM) | | |
| Antisense primer without | 0.032 μl | 3.328 μl |
| fluorescence label (10 μM) | | |
| EX Taq | o.1 μl | 10.4 μl |
| ddH₂O | 5.78 μl | 601.12 μl |
| DNA | 2 μl | |

| | | | | | |
|---|---|---|---|---|---|
| Procedure: | 95° C. | 2 min | | | |
| | 94° C. | 20 s | | | |
| | 65° C. | 30 s | | {close oversize brace} | 35 cycles |
| | 72° C. | 1.5 min | (varies according to | | |
| | | | different fragments) | | |
| | 72° C. | 5 min | | | |
| | 95° C. | 10 min | | | |
| | 70° C. | 20 s | (−0.3° C./ | | |
| | | | cycle) 70 cycles | | |
| | 15° C. | 5 min | | | |

PCR product was placed in dark and on ice after PCR reaction.

5. Enzyme Digestion

PCR product of each DNA pool obtained by the above 4 was digested with CEL I enzyme to obtain products of enzyme digestion. The system of enzyme digestion was shown in Table 8:

TABLE 8
System of enzyme digestion of CEL I

| Master mix | 15 ul for each reaction | ×104 |
|---|---|---|
| PCR products | 9 ul | |
| CEL I | 0.1 ul | 10.4 ul |
| 10 × CEL I Buffer | 1.5 ul | 156 ul |
| ddH₂O | 4.45 ul | 462.8 ul |

Procedure: Enzyme digestion at 45° C. for 15 minutes.

6. Electrophoresis Analysis

1) Electrophoresis

Enzyme-digested products of each PCR product corresponding to each DNA pool obtained by the above 5 were purified and electrophoresed, wherein a sample in each lane was an enzyme-digested product of each PCR product of a DNA pool.

Purification: 15 ul enzyme digested product, 20 ul ddH2O, 5 ul 0.225M EDTA and 60 ul isopropanol were added into a 96 wells plate. The plate was covered by a silica gel cover and the mixture was turned upside down for 30 times and then incubated at room temperature for 15 minutes followed by centrifugation at 4° C., 3000 rpm for 30 minutes. Supernatant was discarded after centrifugation and the 96 wells plate was inverted on a paper towel followed by brief centrifugation at 4° C., 3000 rpm for 10 seconds. 100 ul 75% ethanol was added to the precipitate in the 96 wells plate. The plate was covered by a silica gel cover and the mixture was turned upside down for 30 times followed by centrifugation at 4° C., 3000 rpm for 20 minutes. Repeat the steps twice. The sample plate was dried in a ventilation system for 2 min. The precipitate was dissolved in 5 ul loading buffer after it was free of ethanol and shaked for 5 seconds on a vortex followed by brief centrifugation for 10 seconds. The DNA was denaturated at 85° C. for 10 minutes.

Electrophoresis:

| | |
|---|---|
| 20 ml | 6% gel (commercial available gel) |
| 100 ul | AP (−20° C.) |
| 25 ul | TEMED (4° C.) mix immediately |

The sample was denatured at 85° C. for 10 minutes and then placed on ice for 10 minutes.

TABLE 9
Electrophoresis condition

| | Pre-run | Run |
|---|---|---|
| Volts (v) | 1500 | 1500 |
| Current (mA) | 40 | 40 |
| Power (w) | 40 | 40 |
| Time | 10 min | 3 hr |
| Temperature (° C.) | 45 | 45 |

0.45 ul of each sample was added to a 100 wells paper comb rapidly within 10 minutes of prerunning. 1 ml 1% Ficoll was added into each comb well without TBE and the comb with samples was inserted into the wells rapidly. Li-COR 4300 was turned on and ran for 3 hours. The runtime varies according to the size of amplified fragments.

2) Data Analysis

Photos of the above electrophoresis results were processed by Adobe Photoshop 8.0. Mode was changed from 16 channels to 8 channels. The photos were rotated and set as pictures having a width of 20 cm and a length of 27 cm. Defined ratio was canceled and then brightness and contrast were adjusted. The photos were finally saved in JPEG format and analyzed using Gelbuddy. The photos were observed under 682 nm and 782 nm.

Enzyme-digested products of each DNA pool were analyzed by electrophoresis to identify fertility-lowered mutants or sterile mutants. If enzyme-digested products of said DNA pool generated bright dots under all wavelengths of different fluorescently labeled probes, individual plants of M2 (with number n) of said DNA pool contained or might contain fertility-lowered mutants or sterile mutants; If enzyme-digested product of said DNA pool did not generate bright dots under all wavelengths of different fluorescently labeled probes, individual plants of M2 (with number n) of said DNA pool did not contain or might not contain fertility-lowered mutants or sterile mutants. Said fertility-lowered mutants were plants whose fertility were lowered than said target plant.

The aforementioned method can be used directly to identify a point mutation in a triterpene synthase encoding gene. If enzyme-digested product of said DNA pool generated bright dots under all wavelengths of different fluorescently labeled probes, the triterpene synthase encoding gene in said DNA pool had or might have a point mutation. If enzyme-digested product of said DNA pool did not generate bright dots under all wavelengths of different fluorescently labeled probes, the triterpene synthase encoding gene in said DNA pool did not have or might not have a point mutation.

Partial results are shown in FIG. 3, wherein the left picture represents result of DY-682 and the right picture represents result of DY-782. The arrows point to mutants. Arrow 1 points to one mutant, while arrow 2 points to another mutant. It can be seen that both enzyme-digested products of two DNA pools in two lanes generate bright dots under DY682 and DY782 which indicates that 4 individual plants of M2 of these two DNA pools have sterile mutants or fertility-lowered mutants.

In order to further identify which individual plant was a mutant, 4 individual plants of anyone of the above DNA pools which were identified as sterile mutants or fertility-lowered mutants were used as templates with genomic DNA (mixed with equal quantity) of wild-type rice respectively. Steps 4-5 were repeated to obtain enzyme-digested products of M2 individual plant. Enzyme-digested products of M2 individual plant were purified and electrophoresed, wherein a sample in each lane was an enzyme-digested product of each PCR products of genomic DNA of each M2 individual plant; each lane corresponded to genomic DNA of a M2 individual plant.

If enzyme-digested products of said M2 individual plant generated bright dots under all wavelengths of different fluorescently labeled probes, said M2 individual plant was or might be a sterile mutant or fertility-lowered mutant. If enzyme-digested products of said M2 individual plant did not generate bright dots under all wavelengths of different fluorescently labeled probes, said M2 individual plant was not or might not be a sterile mutant or fertility-lowered mutant.

The specific primer pair corresponding to the enzyme-digested products of PCR product of said lane was identified simultaneously (according to the specific primer pair corresponding to the enzyme-digested product of PCR product of the lane in which bright dots generated, the corresponding specifically amplified product was identified) and used as verification primers subsequently.

Total 3 fertility-lowered mutants of M2 individual plants named P34E8, 4928 and 1708 were selected. Said mutants were also mutants of triterpene synthase encoding gene OsOSC8. The corresponding gene-specific primer pairs of the mutants were as follows: primer pair 2 for selection of P34E8 and 4928 and primer pair 3 for selection of 1708.

II Verification of Fertility-Lowered Lines Selected by TILLING Technology

1. Molecular Verification of Point Mutation of Triterpene Synthase Encoding Gene

RNA was extracted from the above selected 3 fertility-lowered mutants of M2 individual plants P34E8, 4928 and 1708 and reverse-transcribed into cDNA. PCR amplify the cDNA with primer pair 1. The PCR products were sequenced and the mutated sites of the 3 mutants P34E8, 4928 and 1708 were identified as shown in the following table 10:

TABLE 10
Mutated sites of each mutant and change of amino acid

| | Change of | Change of |
|---|---|---|
| Mutant No. | Nucleotide | Amino Acid |
| P34E8 | G764A | W255- |
| 4928 | G809A | G270E |
| 1708 | G1431A | R477K |

Sites of amino acid and nucleotide in the above table correspond to the sites of the sequences of protein (amino acid sequence as shown in SEQ ID NO.2) and gene (nucleotide sequence as shown in SEQ ID NO.5) of OsOSC8.

Amino acid sequence of P34E8 was obtained by mutating the amino acid residue at the 255 position of N′ end of the sequence as shown in SEQ ID NO.2 from Trp to termination codon. Nucleotide sequence of P34E8 was obtained by mutating the nucleotide residue at the 764 position of 5′ end of the sequences shown in SEQ ID NO.5 from G to A.

Amino acid sequence of 4928 was obtained by mutating the amino acid residue at the 270 position of N′ end of the sequence as shown in SEQ ID NO.2 from Gly to Glu. Nucleotide sequence of 4928 was obtained by mutating the nucleotide residue at the 809 position of 5′ end of the sequence as shown in SEQ ID NO.5 from G to A.

Amino acid sequence of 1708 was obtained by mutating the amino acid residue at the 477 position of N′ end of the sequence as shown in SEQ ID NO.2 from Gly to Lys. Nucleotide sequence of 1708 was obtained by mutating the nucleotide residue at the 1431 position of 5′ end of the sequence as shown in SEQ ID NO.5 from G to A.

The mutant P34E8 selected above was a mutant strain of OsOSC8 which was deposited in China General Microbiological Culture Collection Center (CGMCC for short, address: No. 3, Courtyard No. 1, West Road Beichen, Chaoyang District, Beijing) on 28 May 2012 with a deposit Number of CGMCC No. 6150. The classification and nomenclature is rice Oryza sativa.

2. Phenotype Identification of Sterile Lines Selected by TILLING

Seeds of M3 of the 3 fertility-lowered mutants P34E8, 4928 and 1708 were used in the following experiments.

1) Statistics of Setting Percentage of the Mutants

(1) Seeds of the 3 fertility-lowered mutants P34E8, 4928 and 1708 obtained in the above I were planted at 18° C./25° C. (night/day) in glass green house with natural lighting. Growth conditions: temperature 18° C./30° C. (night/day), humidity 30%-50%, natural lighting.

(2) Setting percentage of florets can be observed 2 weeks after rice flowering; 5 panicles were analyzed and experiments were repeated 3 times. The results were mean value±standard deviation. The control was wild-type rice.

As a result, setting percentage of wild-type rice is 94.81%±1.34%; setting percentage of mutant P34E8 is only 1.85%±0.49%; setting percentages of mutant 4928 and mutant 1708 are 4.38%±0.24% and 3.87%±0.36% respectively. The results indicate that the fertility of selected mutants P34E8, 4928 and 1708 is lower than the wild-type rice.

2) Fertility Phenotype Identification of Mutants

Seeds of the fertility-lowered mutant P34E8 (S6) obtained in the above I was planted at 18° C./25° C. (night/day) in glass green house with natural lighting. Growth conditions: temperature 18° C./30° C. (night/day), humidity 30%-50%, natural lighting.

(1) Tillering Mutant P34E8 and wild-type rice were observed during vegetative growth stage (50-70 days after seeding). Both of them grew normally and had 3-5 tillers, wherein mutant P34E8 did not show abnormal characters.

(2) Floral Organ

14 weeks after seeding, mutant P34E8 (S6) and wild-type rice began heading and flowering. Inflorescence, shape of florets and number and size of floral organs of mutant P34E8 and wild-type rice were observed.

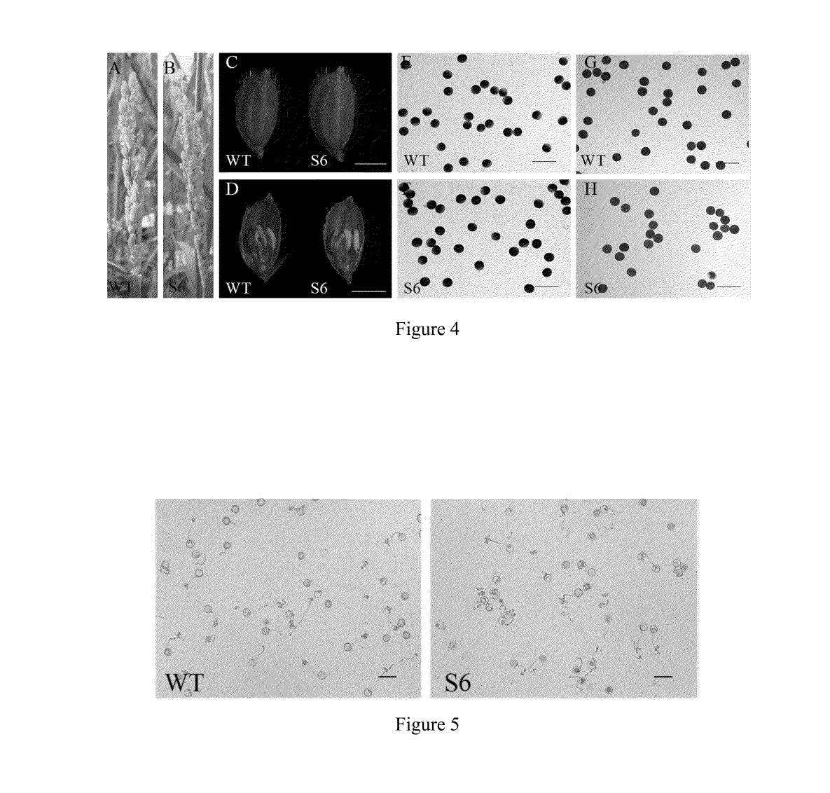

As a result, mutant P34E8 (S6) had normal panicles and oblong florets with a complete set of floral organs which contained one lemma, one glumelle, six stamens, one pistil (with a two-split feathery stigma) and two lodicules. The size of floral organs of mutant P34E8 (S6) developed normally (FIG. 4 A, B, C, D) and didn't have changes compared to wild-type rice.

I2-KI staining (KI 3 g, I₂ 1 g, diluted to 300 ml) of pollens of mutant P34E8 (S6) and wild-type rice were carried out and observed under a microscope (microscope model: OLYMPUS BX51) after 5 minutes. The result of staining showed that pollens of the mutant and wild-type rice were blue black in I₂ which indicated that accumulation of starch thereof was normal (FIG. 4 E, F).

Alexander staining (refer to Alexander M P., 1969, Stain Technol, 44:117-122) of pollens of mutant P34E8 (S6) and wild-type rice were carried out. The staining solution was prepared as 50× Master solution. 10 ml absolute ethanol, 1 ml 1% malachite green (prepared with 95% ethanol), 5 g phenol, 5 g chloral hydrate, 5 ml 1% acid fuchsin solution, 0.5 ml 1% orange G aqueous solution, 2 ml glacial acetic acid and 25 ml glycerol were mixed and adjusted to 100 ml with distilled water and stored in a brown bottle. The Master solution was diluted before use according to a ratio of Master solution:distilled water=3:47 (v:v). Staining method was the same to I2-KI staining. The result of staining showed that pollens of the mutant and wild-type rice were dyed purple red which indicated that pollens were viable (FIG. 4 G, H).

In vitro germination of pollens of mutant P34E8 (S6) and wild-type rice were carried out to detect the vitality thereof. In particular, pollens were cultivated in a culture medium containing 20% sucrose, 10% PEG4000, 40 mg/L boric acid, 3 mmol/L calcium nitrate, 3 mg/L vitamin B1. Detection Method: 2-3 drops of culture medium were dropped on a glass slide, and then anthers of a floret which just opened and was about to loose powder were placed in the culture medium and broken by a pointed tweezer. Massive anther walls were removed and the sample was covered with a coverslip. Samples were placed in a big culture dish covered with wet gauze (moisturizing) and incubated at 30° C. in an incubator and observed after 30 minutes.

The result of observation indicated that the highest pollen germination rate of wild-type rice was 82.8% and the highest pollen germination rate of mutant P34E8 (S6) was 80%. Therefore, vitality of pollens of mutant was good in in vitro germination experiment (FIG. 5).

(3) Ability of Pollens to Adhere to Stigmas

In vivo germination of pollens of mutant P34E8 (S6) and wild-type rice were carried out to detect the vitality on stigmas thereof. Callose is β-3-1,3-glucan which is usually distributed in sieve tube, newly formed cell walls, pollens and pollen tubes of higher plants. It can give out yellow to yellow-green fluorescence under UV excitation after dyed with water-soluble aniline blue. Therefore, germination of pollens on stigmas, status of pollen tube development, as well as state of deposition of callose on the surface of stigmas, etc. can be observed by placing pollinated ovaries after dyed with aniline blue under fluorescence microscope and then to determine whether the pollens were compatible with the stigmas. Method (Refer to Endo M et al., 2009, Plant Cell Physiol, 50:1911-1922): Rice florets at different time after pollination were fixed in Kano's stationary liquid (absolute ethanol:glacial acetic acid=3:1 (v:v)). The volume of the fixation solution is at least 10 times of the materials. Fix for 30 minutes to 2 hours (should not over 24 hours, or the tissue will become fragile). Then rinse the materials successively by 95% ethanol for 5 minutes, 70% ethanol for 5 minutes and distilled water for 5 minutes to remove glacial acetic acid. Then florets were treated with 1N NaOH at 60° C. for 30 minutes and the softened materials were rinsed by distilled water 3 times (to remove most sodium hydroxide. As the materials were brittle now, carefully operate), each time for 5 minutes. 0.1% water-soluble aniline blue dye solution (Aniline blue, Sinopharm Chemical Reagent Co., Ltd., prepared with 0.1M potassium phosphate aqueous solution) was dropped after rinse. Materials were just immerged in dye solution and dyed in dark for 1 hour or so. A drop of 50% glycerol was dropped on a slide. Florets after dyeing were taken out by a tweezer. Stigmas were isolated. Two-split feathery stigmas were placed roughly straight and covered by a coverslip. Styluses were spreaded by gentle pressure (not too hard) and then observed under UV Fluorescence microscope (Microscope model: OLYMPUS BX51).

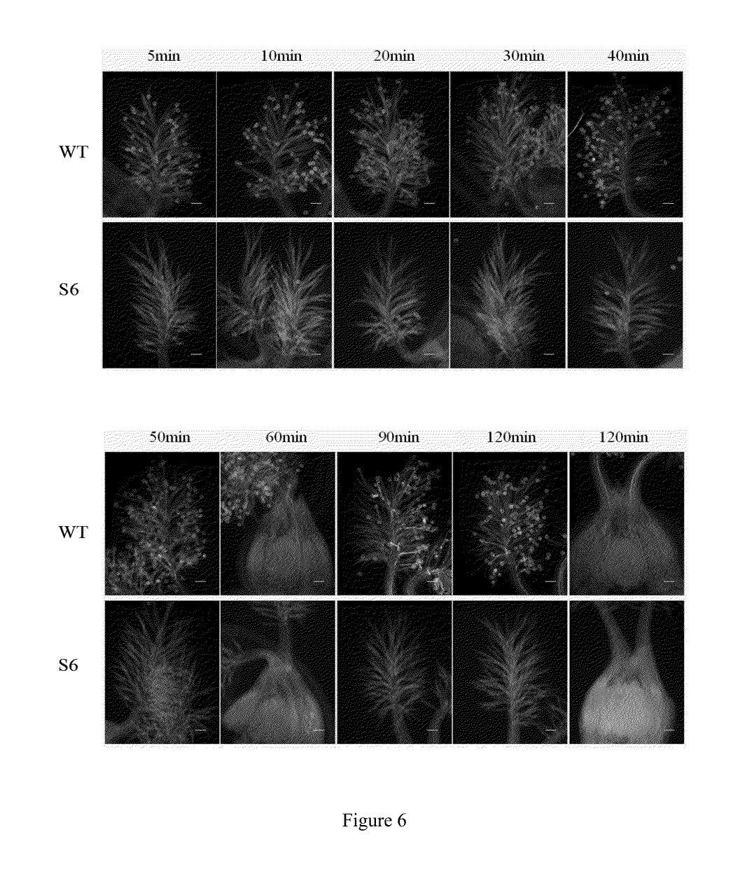

The result of observation was shown in FIG. 6. It indicated that 5 minutes after pollination patial pollens of wild-type rice adhered to stigmas and more and more pollens adhered to stigmas with time extending. 20 minutes after pollination pollen tubes have began to extend and 60 minutes after pollination some pollen tubes have entered the ovule. However, in the corresponding time, little or no pollens of mutants adhered to stigmas, which further proved that reduction of fertility of mutant P34E8 (S6) was caused by reduction of ability of pollens to adhere to stigmas or the fact that pollens did not adhere to stigmas.

In order to prove that the condition that pollens of mutant P34E8 (S6) did not adhere to stigmas was caused by pollen grain itself or by changes of stigmas, mutant P34E8 (S6) was hybridized with wild-type. Uncracked pollens of wild-type were pollinated on stigmas of mutant and uncracked pollens of mutant were pollinated on stigmas of wild-type. 20 minutes and 60 minutes after pollination stigmas were fixed in Kano's fixation solution and stained with aniline blue. Adhesion and germination of pollens on stigmas were detected.

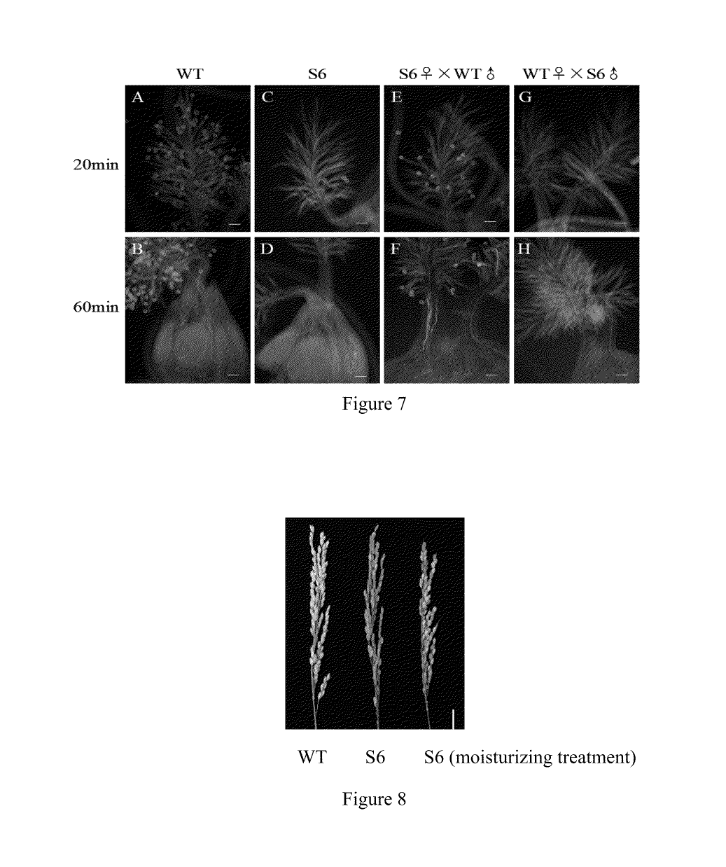

The result of detection was shown in FIG. 7. It indicated that pollens of wild-type could adhere to stigmas of homozygous mutant, germinate and enter the ovule, while pollens of mutant could not adhere to stigmas of wild-type. This indicated that the pollens of mutant failing to adhere to stigmas was caused by pollen itself rather than stigmas.

The above experiments further proved that fertility of mutant P34E8 (S6) was lowered compared to wild-type rice and it was caused by reduction of ability of pollens to adhere to stigmas or the fact that pollens did not adhere to stigmas.

Mutants 4928 and 1708 were identified using the same method and the result had no significant difference with that of mutant P34E8 (S6). Fertility of both mutants was lowered than wild-type rice, and reduction of fertility was caused by the fact that pollens did not adhere to stigmas.

The above results indicate that mutation of triterpene synthase encoding genes can result in reduction of rice fertility. Thus, TILLING selection method can be used to obtain fertility-lowered rice, even sterile rice.

### Example 3

### Restoring Fertility or Improving Fertility

Seeds of M3 generation of fertility-lowered mutant P34E8 were used in the following experiments.

Moisturizing treatment: T0 generation of RNA interference transgenic rice of RNAi-9, RNAi-12, RNAi-20 and RNAi-21 obtained in the above example 1 and mutant P34E8 (S6) obtained in example 2 were seeded. During rice anthesis (anthesis is from the 80^{th} day to the 100th day after seeding), humidity for growth of rice inflorescence was maintained at 80-100% and returned to natural humidity (natural humidity was 40-60%) after one week. Specifically, the method of maintaining humidity for growth of rice inflorescence was as follows: Wrapping the whole rice inflorescence with a plastic bag (standard: length×width=27 cm×15 cm) and clipping the plastic bag at the opening with a paper clip; or using preservative film to cover the whole inflorescence. There was no need to use a paper clip to clip preservative film since preservative film sticked together easily.

Rice inflorescence flowered from the top to the bottom and the anthesis of the whole inflorescence was about one week. Wrapping plastic bag and preservative film must be removed in time after flowering of the whole inflorescence because a lot of vapor was gathered during wrapping and too high humidity was not good for the following fructification.

Setting percentages of 5 panicles were analyzed and the experiment was repeated 3 times. The results were mean value±standard deviation. The control was wild-type rice ZH11 (WT).

Setting percentages of wild-type rice ZH11 and T0 generation of RNA interference transgenic rice of RNAi-9, RNAi-12, RNAi-20 and RNAi-21 obtained in example 1 after moisturizing treatment were shown in FIG. 2. Setting percentages of wild-type rice ZH11 and T0 generation of RNA interference transgenic rice of RNAi-9, RNAi-12, RNAi-20 and RNAi-21 obtained in example 1 after moisturizing treatment were 89.9±1.8%, 78.9±11.3%, 60.2±3.9%, 85.2±16.18% and 80.3±3.9% respectively; In contrast, the setting percentages of RNAi-9, RNAi-12, RNAi-20 and RNAi-21 without moisturizing treatment were only 42.1±15.3%, 37.9±3.9%, 18.6±16.1 and 81.1±6.1%, respectively.

Fruiting phenotype of mutant P34E8 (S6) after moisturizing treatment was shown in FIG. 8. It can be seen in the figure that setting percentage of P34E8 after moisturizing treatment was greatly increased. Statistical analysis of the setting percentage showed that setting percentage of mutant P34E8 after moisturizing treatment could be 76.25%±3.88%, whereas setting percentage of mutant P34E8 without moisturizing treatment was only 1.85%±0.49%. It indicated that maintaining humidity of rice inflorescence growth could restore fertility or improve fertility.

### Example 4

### Use of Fertility-Lowered Mutant P34E8 (S6) in Breeding

Seeds of M3 of fertility-lowered mutants 4928 and P34E8 (S6) were used in the following experiments.

Preparation of hybrid rice seeds: Grouping for production of hybrid seeds of fertility-lowered mutants 4928 and P34E8 (S6) obtained in example 2 and wild-type rice ZH11 and rice 9311(Jun Yu, Songnian Hu, Jun Wang, Gane Ka-Shu Wong, . . . Jian Wang, Lihuang Zhu, Longping Yuan, Huanming Yang. A draft sequence of the rice (Oryza sativa ssp. indica) genome. Science. 296: 79-92, 2002; the public can obtain the material from Institute of Botany, the Chinese Academy of Sciences.) was carried out respectively. Each group contained 30 mutant plants and appropriate amount of wild-type plants were planted at intervals to provide pollens, with 3 replicates.

Meanwhile, fertility-lowered mutants 4928 and P34E8 (S6) were planted respectively in a separate group for selfing. Each group contained 30 plants, with 3 replicates, as control. Artificial supplementary pollination was performed during flowering period (2012 Aug. 20-2012 Sep. 1). The method specifically was: Using an about 2 meters long bamboo pole to pat inflorescence of wild-type rice 5-10 times, to make pollens fly towards inflorescence of mutants, twice a day, at 11:00 and 13:00; the control group was not treated.

After maturation, 30 plants of each hybrid line and selfed line were selected. Setting percentage of one main spike of each plant was analyzed.

The result was shown in Table 11:

TABLE 11
Setting percentage

| | 9311 | ZH11 | 4928 | P34E8 |
|---|---|---|---|---|
| 4928 | 28.89 ± 3.42% | 31.81 ± 2.79% | 7.45 ± 1.38 | — |
| P34E8 | 22.32 ± 1.71% | 31.34 ± 1.86% | — | 12.84 ± 1.66 |

As seen from the table, selfing setting percentages of mutant 4928 and P34E8 (S6) after in field were 7.45 and 12.84 and hybridization setting percentages were 22.32%-31.81%. Mu yield of production of hybrid seeds was approximately 100-150 kilograms. It indicates that mutants can be used in hybrid rice breeding.

### Example 5

### Homologous Genes of OsOSC8 from Other Plants and Prediction of Their Functions

I Cloning Homologous Genes

1) Obtaining of TaOSC1 (from Wheat) and HvOSC1 (from Barley)

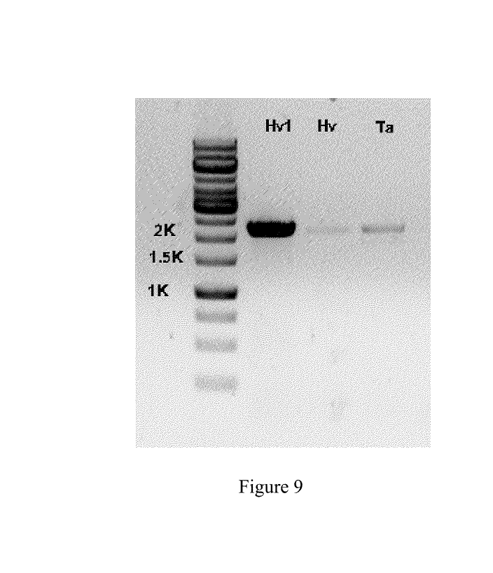

Primer design according to EST sequences of hexaploid wheat: 5′primer: 5′-ATGTGGAAGCTCAAGATCGC-3′ (SEQ ID NO.12); 3′ primer: 5′-TTAGCCAGAGCAAAGTACTAAT-3′ (SEQ ID NO.13). Total RNA of flowers of Chinese spring wheat (Triticum aestivum L. Jizeng Jia, Zhengbin Zhang, K. Devos, M. D. Gale. Analysis of genetic diversity of 21 chromosomes of Triticum aestivum L. based on RFLP mapping sites. Science in China Series C: Life Sciences. 2001(01); The public can obtain the material from Institute of Botany, the Chinese Academy of Sciences) and barley “Varda” (Hordeum vulgare L. Qi X, Niks R E, Stam P, Lindhout P. 1998. Identification of QTLs for partial resistance to leaf rust (Puccinia hordei) in barley. Theor Appl Genet, 96: 1205-1215; The public can obtain the material from Institute of Botany, the Chinese Academy of Sciences.) was used as templates to perform RT-PCR amplification to obtain cDNA. PCR products were detected by 0.8% agarose gel electrophoresis after the reaction finished. The results of detection were shown in FIG. 9. The left lane was DNA standard (1 kb Ladder), lane Ta was RT-PCR product of wheat, and lanes Hv1 and Hv were RT-PCR products of barley. Bands with a molecular weight of 2-3 kb were obtained and they are consistent with expected size.

RT-PCR products of wheat and barley were sequenced and the results were as follows:

Gene of RT-PCR product of wheat was named TaOSC1, having a nucleotide sequence as shown in SEQ ID NO.14 which consists of 2280 bases. The open reading frame (ORF) thereof comprises the 1-2280 bases of 5′ end and the protein encoded by the gene was TaOSC1. Amino acid sequence of the protein was shown in SEQ ID NO.15. Homology comparison between TaOSC1 and OsOSC8 showed that similarity of the nucleotide and amino acid sequences were 84.32% and 85.18% respectively.

Gene of RT-PCR product of barley was named HvOSC1, having a nucleotide sequence as shown in SEQ ID NO.16 which consists of 2280 bases. The open reading frame (ORF) thereof comprises the 1-2280 bases of 5′ end and the protein encoded by the was HvOSC1. Amino acid sequence of the protein was shown in SEQ ID NO.17. Homology comparison between HvOSC1 and OsOSC8 showed that similarity of the nucleotide and amino acid sequences were 81.58% and 81.35% respectively.

2) SrOSC1 from sorghum (sorghum bicolor L.) and ZmOSC1 from maize (zea may L.)

Functions of homologous genes of OsOSC8 in gramineous plants might be very conservative, so putative coding sequences of homologous genes of OsOSC8, namely SrOSC1 and ZmOSC1 were obtained according to the whole genome sequences (http://phyto5.phytozome.net/) of sorghum (sorghum bicolor L.) and maize (zea may L.).

Amino acid sequence of protein SrOSC1 from sorghum (sorghum bicolor L.) was shown in SEQ ID NO.18 and the gene encoding the protein was shown in SEQ ID NO.19.

Amino acid sequence of protein ZmOSC1 from maize (zea may L.) was shown in SEQ ID NO.20 and the gene encoding the protein was shown in SEQ ID NO.21.

The above genes can be obtained by artificial synthesis.

II Prediction of Functions

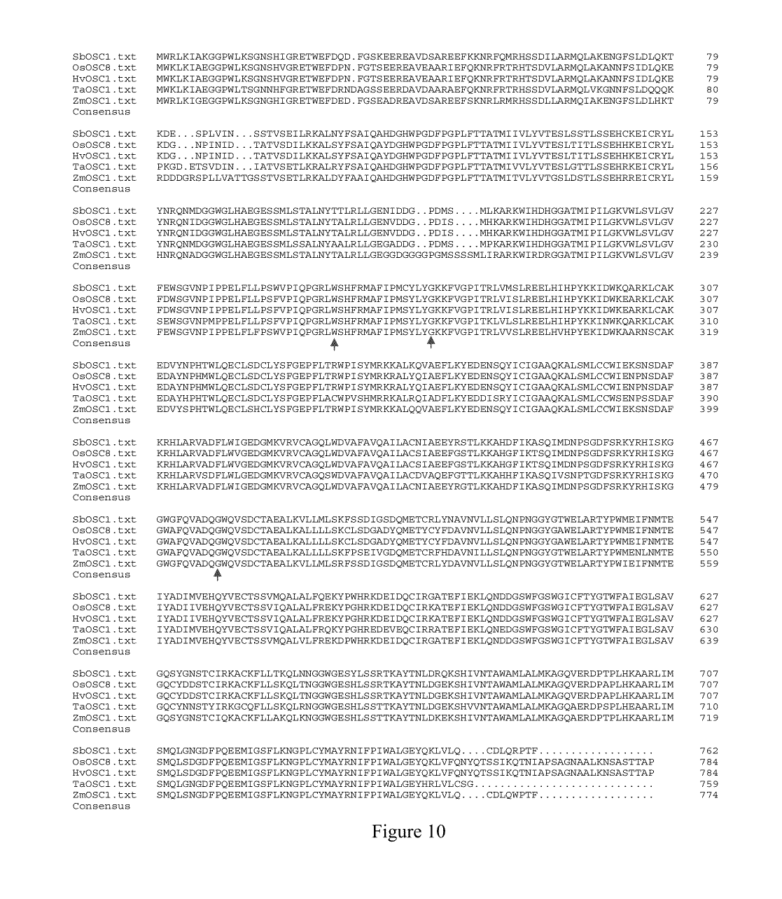

TaOSC1 (from wheat), HvOSC1 (from barley), SrOSC1 (from sorghum) and ZmOSC1 (from maize) obtained above were aligned and the result was shown in FIG. 10. Red arrowheads show the mutated sites of P34E8, 4928 and 1708. Mutation of these sites in sorghum, maize, wheat and barley may lead to similar restorable sterile phenotype. It can be seen that the method can be used in crossbreeding of sorghum, maize, wheat and barley.

It proved that functions of homologous genes of OsOSC8 in gramineous plants are very conservative.

Thus, according to the studies on OsOSC8 from rice in preceding examples, it can be inferred that silencing the genes of TaOSC1 (from wheat), HvOSC1 (from barley), SrOSC1 protein (from sorghum) and ZmOSC1 protein (from maize) which have high homology with OsOSC8 can also obtain sterile lines.

### INDUSTRIAL APPLICABILITY

The experiments of the present invention prove that the present invention provides various methods for preparing sterile lines or fertility-lowered lines, including RNA interference or TILLING (Targeting Induced Local Lesions IN Genomes) technology selection. These methods are achieved by silencing expression of gene encoding triterpene synthase. The present invention also provides methods for restoring or improving fertility. Sterile lines prepared by the methods of the present invention establish the basis of rice heterosis and crossbreeding.

1. An RNA interference vector obtained by inserting the DNA molecule as shown in SEQ ID NO.1 into a pH7GWIWG2(II) vector.

2. The RNA interference vector according to claim 1, wherein said RNA interference vector is obtained by inserting the DNA molecule as shown in SEQ ID NO.1 into a pH7GWIWG2(II) vector by means of homologous recombination.

3. The RNA interference vector according to claim 1 or 2, wherein said RNA interference vector is prepared according to the following method:
1) obtaining an intermediate vector by BP reaction between the DNA molecule as shown in SEQ ID NO.1 and a pDONR221 vector;
2) obtaining an RNA interference vector by LR reaction between said intermediate vector and a pH7GWIWG2(II) vector;
wherein said DNA molecule as shown in SEQ ID NO.1 is prepared according to the following method: Using rice cDNAs as a template and primer pair A to carry out PCR amplification, the obtained PCR product is the DNA molecule as shown in SEQ ID NO.1;
said primer pair A consists of the single chain DNA as shown in SEQ ID NO.3 and the single chain DNA as shown in SEQ ID NO.4.

4. A recombinant bacteria or transgenic cell line comprising said RNA interference vector of any one of claims 1 to 3.

5. Use of said RNA interference vector of any one of claims 1 to 3 and said recombinant bacteria or transgenic cell line of claim 4 in cultivating rice sterile lines or reducing rice fertility.

6. A method of cultivating a transgenic plant, said method comprising obtaining a transgenic plant by introducing said RNA interference vector of any one of claims 1 to 3 into a target plant; wherein said transgenic plant comprises the following 1) or 2):
1) Sterile transgenic plant; or 2) The fertility of said transgenic plant is lower than that of said target plant;
wherein said target plant is monocot plant; said monocot plant is rice.

7. A transgenic plant obtained by the method of claim 6; wherein said transgenic plant is a sterile transgenic plant or fertility-lowered transgenic plant; said plant is monocot plant; said monocot plant is rice.

8. A method for cultivating a target plant to a sterile mutant or fertility-lowered mutant, comprising the following steps:
1) Mutating seeds of a target plant; designing primer pair B and fluorescently labeled primer pair B which are used to specifically amplify the gene encoding triterpene synthase from a target plant;
wherein mutated seeds of a target plant are obtained by treating a number of seeds of the target plant with sodium azide;
wherein each primer of said fluorescently labeled primer pair B is labeled with different fluorescently labeled probes; wherein said different fluorescently labeled probes have different wavelengths;
2) Cultivating said mutagenic seeds to obtain the first generation of mutation M1; self-cross of the first generation of mutation M1 is carried out to obtain the second generation of mutation M2;
3) Extracting genomic DNA of individual plant of the second generation of mutation M2; mixing genomic DNA of individual plant (with number n) of M2 to obtain a DNA pool; wherein n is 2-8;
4) Using each of said DNA pool as a template and both said primer pair B and fluorescently labeled primer pair B to obtain PCR products;
5) Digesting said PCR product by endonuclease CELI to obtain enzyme-digested product of a DNA pool;
6) Detecting the enzyme-digested products of each said DNA pool by electrophoresis; wherein if enzyme-digested products of said DNA pool generates bright dots under all wavelengths of different fluorescently labeled probes, individual plant of M2 (with number n) of said DNA pool contain or may contain fertility-lowered mutants or sterile mutants; wherein if enzyme-digested products of said DNA pool does not generate bright dots under all wavelengths of different fluorescently labeled probes, individual plants of M2 (with number n) of said DNA pool do not contain or may not contain fertility-lowered mutants or sterile mutants; wherein said fertility-lowered mutants are plants whose fertility is lower than that of said target plant.

9. The method according to claim 8, wherein following said step 6), said method further comprises the following steps:
genomic DNAs of individual plant (with number n) of M2 which contain or may contain fertility-lowered mutants or sterile mutants are mixed with genomic DNA of said target plant as templates; steps 4)-5) are repeated to obtain enzyme-digested products of individual plant of M2;
wherein each of said enzyme-digested products of individual plant of M2 is detected by electrophoresis; wherein if said enzyme-digested products of individual plant of M2 generate bright dots under all wavelengths of different fluorescently labeled probes, said individual plant of M2 is or may be a sterile mutant or fertility-lowered mutant; wherein if said enzyme-digested products of individual plant of M2 do not generate bright dots under all wavelengths of different fluorescently labeled probes, said individual plant of M2 is not or may not be a sterile mutant or fertility-lowered mutant.

10. The method according to claim 8 or 9,
wherein in step 1), treating a number of seeds of the target plant with sodium azide is immersing a number of seeds of the target plant in an aqueous solution of sodium azide with a concentration of 2 mM for 6 hours;
wherein the amino acid sequence of said triterpene synthase is shown in SEQ ID NO. 2;
wherein said different fluorescently labeled probes are fluorescently labeled probe DY-682 with a wavelength of 682 nm and fluorescently labeled probe DY-782 with a wavelength of 782 nm;
wherein the nucleotide sequence of the gene encoding said triterpene synthase is shown in SEQ ID NO. 5;
wherein said primer pair B is selected from the following group consisting of primer pairs as shown in 1)-3):
1) Primer pair consisting of the single chain DNA molecule as shown in SEQ ID NO. 6 and the single chain DNA molecule as shown in SEQ ID NO. 7;
2) Primer pair consisting of the single chain DNA molecule as shown in SEQ ID NO. 8 and the single chain DNA molecule as shown in SEQ ID NO. 9;
3) Primer pair consisting of the single chain DNA molecule as shown in SEQ ID NO. 10 and the single chain DNA molecule as shown in SEQ ID NO. 11;
wherein aid target plant is a monocot plant; said monocot is a monocot graminaceous plant; specifically, said monocot graminaceous plant is rice.

11. A sterile mutant or fertility-lowered mutant prepared by the method of any one of claims 8 to 10.

12. The fertility-lowered mutant according to claim 11, wherein the Deposit Number of said fertility-lowered mutant is CGMCC NO.6150.

13. Use of a transgenic plant of claim 7 or said sterile mutant or fertility-lowered mutant of claim 11 or said fertility-lowered mutant of claim 12 in the production of hybrid seeds.

14. A method for obtaining a sterile mutant or fertility-lowered mutant, comprising silencing or inactivating the gene encoding triterpene synthase in a target plant; wherein said fertility-lowered mutant is a plant whose fertility is lower than that of the target plant.

15. The method according to claim 14, wherein said target plant is a monocot plant or dicot plant; said monocot plant is specifically a monocot graminaceous plant; said monocot graminaceous plant is rice, wheat, barley, sorghum or maize;
wherein the amino acid sequence of the triterpene synthase of said rice is shown in SEQ ID NO. 2; the nucleotide sequence of the gene encoding triterpene synthase of said rice is shown in SEQ ID NO. 5;
wherein the amino acid sequence of the triterpene synthase of said wheat is shown in SEQ ID NO. 15; the nucleotide sequence of the gene encoding triterpene synthase of said wheat is shown in SEQ ID NO. 14;
wherein the amino acid sequence of the triterpene synthase of said barley is shown in SEQ ID NO. 17; the nucleotide sequence of the gene encoding triterpene synthase of said barley is shown in SEQ ID NO. 16;
wherein the amino acid sequence of the triterpene synthase of said sorghum is shown in SEQ ID NO. 18; the nucleotide sequence of the gene encoding triterpene synthase of said sorghum is shown in SEQ ID NO. 19;
wherein the amino acid sequence of the triterpene synthase of said maize is shown in SEQ ID NO. 20; the nucleotide sequence of the gene encoding triterpene synthase of said maize is shown in SEQ ID NO. 21.

16. The method according to claim 14 or 15, wherein said silencing or inactivating of the gene encoding triterpene synthase in rice is at least one of the following 1)-3):
1) The nucleotide residue at the 764 position of said gene encoding triterpene synthase in rice is mutated from G to A;
2) The amino acid residue at the 809 position of said gene encoding triterpene synthase in rice is mutated from G to A;
3) The nucleotide residue at the 1431 position of said gene encoding triterpene synthase in rice is mutated from G to A.

17. A method for restoring or improving fertility of an original plant, comprising the following step: Maintaining the humidity for growth of plant inflorescence at 80-100% during anthesis of an original plant; said original plant is a sterile mutant or fertility-lowered mutant.

18. The method according to claim 17, wherein said sterile mutant or fertility-lowered mutant is said transgenic plant of claim 7 or said sterile mutant or fertility-lowered mutant of claim 11 or said fertility-lowered mutant of claim 12.

19. The method according to claim 17 or 18, wherein the time period of maintaining the humidity for growth of plant inflorescence is one week.

20. The method according to any one of claims 17 to 19, wherein the method of maintaining the humidity for growth of plant inflorescence is wrapping the whole inflorescence of said original plant;
wherein said wrapping is specifically using a plastic bag to slip over the whole inflorescence or using preservative film to cover the whole inflorescence.
